(19) United States
(12) Patent Application Publication
(10) Pub. No.: US 2007/0184455 A1
(43) Pub. Date: Aug. 9, 2007

(54) Evaluation of spectra

(76) Inventors: Cheryl Arrowsmith, Toronto (CA); Alexander Grishaev, Falls Church, VA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/846,359

(22) Filed: May 14, 2004

Related U.S. Application Data
(60) Provisional application No. 60/471,201, filed on May 16, 2003.

(51) Int. Cl. C12Q 1/68; G06F 19/00

(52) U.S. Cl. 435/6; 702/20

(57) ABSTRACT

Systems, methods, products and analyzers that allow evaluation of spectra of molecules, including proteins, nucleic acids and small molecules, are provided. The spectra that may be evaluated by the systems, methods, products and analyzers include, for example, spectra collected by the techniques of NMR, mass spectrometry, infrared and RAMAN spectroscopy, chromatography, etc.

### RELATED APPLICATIONS

This application claims the benefit of priority to U.S. Provisional Patent Application No. 60/471,201, filed on May 16, 2003, which application is hereby incorporated by reference in its entirety.

### INTRODUCTION

Genomic sequence information for many organisms is now available. However, knowledge of the complete genomic sequence is only the first step towards understanding the function of encoded proteins and nucleic acid molecules. Structural information acquired on a genome wide level in all likelihood will provide valuable information for predicting the rules that govern the formation of secondary and tertiary structure in proteins and nucleic acids. Such insight should prove useful in understanding the biochemical function of molecules and should provide an opportunity for rapid progress in the identification of targets for therapeutics.

Availability of sequence information makes it possible to isolate biological molecules for structural determination. Several high throughput purification methods are now available to clone, express and purify proteins from an entire genome. Such methods can also be adopted to purify several fragments or mutants of the same or different proteins. The use of recombinant methods allow necessary modifications to the native proteins in order to facilitate purification as well as make samples appropriate for structural analyses, for example, by labeling the protein (e.g., with isotopic labels, polypeptide tags, etc.) or by creating fragments of the polypeptide, such as those corresponding to functional domains of a multi-domain protein.

One research challenge is to determine samples and/or solution conditions amenable for analysis by a particular structural method. For example, structural determination by Nuclear Magnetic Resonance (NMR) spectroscopy may be limited by the size and solubility properties of a sample. Moreover, it is known that, in certain instances, even small changes in the amino acid sequence of a protein may lead to dramatic affects on protein solubility. Determining samples appropriate for analysis by NMR spectroscopy may be accomplished by collecting and analyzing spectra for particular spectroscopic properties. Such evaluations can be conducted manually for a limited number of spectra and are conventionally applied before pursuing complete 3D structural determination by NMR or for screening the binding of small molecules to proteins or nucleic acids.

When using spectroscopic techniques to screen samples on a genomic level or screen libraries of compounds for structural characteristics or ability to bind a target, scanning and evaluating the vast number of spectra necessitates the use of automated techniques.

### SUMMARY OF THE INVENTION

In part, the present disclosure is directed towards methods of evaluating spectra of biological molecules.

In part, this disclosure is directed to systems, methods, products and analyzers that allow evaluation of spectra of molecules, including proteins, nucleic acids and small molecules. The spectra that may be evaluated by the systems, methods, products and analyzers include, for example, spectra collected by the techniques of NMR, mass spectrometry, infrared and RAMAN spectroscopy, chromatography, etc.

In part, this disclosure is directed to a method of evaluating one or more spectra comprising providing a training set based on a plurality of spectra, associating the spectra of the training set based on the attributes of at least two spectral parameters with at least two categories, scoring the spectral parameters of the spectra of the training set in the categories, comparing the spectral parameters of one or more sample spectra to the scored spectral parameters of the training set and classifying the sample spectra into one of the categories based on the comparison. In certain embodiments this disclosure is also directed to a method of evaluating one or more spectra comprising providing a training set based on a plurality of spectrum, associating the spectra of the training set based on the attributes of at least two spectral parameters with at least two categories, scoring the spectral parameters of the spectra of the training set in the categories. In other aspects, this disclosure is further directed to a method of evaluating one or more sample spectra comprising obtaining a training set of a plurality of spectrum scored by the attributes of at least two or more spectral parameters in two or more categories, comparing the spectral parameters of one or more sample spectra to the scored spectral parameters of the training set and classifying the sample spectra into one of the categories based on the comparison.

In part, this disclosure is directed to a computer product for evaluating one or more sample NMR spectra, the computer product disposed on a computer-readable medium and having instructions for causing a processor to score attributes of at least one spectral parameter of one or more NMR spectra associated with one or more categories of a training set, compare the one or more spectral parameters of the one or more sample NMR spectra to the scored spectral parameters of said training set and classify the one or more sample NMR spectra into one of the categories.

Further embodiments of the present invention are described in the claims appended hereto, which are incorporated by this reference in their entirety. The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE FIGURES

FIG. 1 is a schematic representing a method for evaluating a plurality of spectra.

FIG. 2 shows exemplary two-dimensional (¹H, ¹⁵N)HSQC spectra of a training set of NMR spectra. Spectra for proteins 1 and 2 are classified as good, proteins 3 and 4 are classified as promising, proteins 5 and 6 are classified as unfolded and proteins 7 and 8 are classified as poor.

FIG. 3 outlines an exemplary algorithm that can be used to evaluate NMR spectra using the spectral parameters of chemical shift, number of peaks observed versus number expected, peak width and peak intensity.

FIG. 4 shows exemplary two-dimensional (¹H, ¹⁵N) HSQC sample spectra classified (using the algorithm outlined in FIG. 3) into the categories of (a) good, (b) promising, (c) unfolded and (d) poor.

FIG. 5 shows Table 2 which presents the results of the evaluation of the spectra from Example 2.

### DETAILED DESCRIPTION OF THE INVENTION

1. Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing.

The term “attribute” refers to a feature of a spectral parameter. The attributes of a spectral parameter will vary with the type of spectrum. For example, for the spectral parameter of peak shape in an NMR spectrum, attributes may be the width of a peak, the height of a peak, the volume of a peak, etc. In another example, for the spectral parameter of number of peaks observed in an NMR spectrum, an attribute may be the fraction of peaks observed of the total number expected for a molecule of interest. In yet another example, for the spectral parameter of peak location in an NMR spectrum, an attribute may be a chemical shift or a range of chemical shifts expected for a peak. In another example, for the spectral parameter of number of peaks observed in a spectrum acquired by mass spectrometry, an attribute may be a pattern of peaks expected for a molecule of interest. In a further example, for the spectral parameter of peak location in a spectrum acquired by mass spectrometry, attributes may be molecular mass and charge. In yet another example, for the spectral parameter of peak intensity in infrared or RAMAN spectroscopy, attributes may be the width of a peak, the height of a peak, the volume of a peak, etc. Other suitable attributes for various types of spectrum will be known to those of skill in the art.

One or more attributes of a spectral parameter may be indicative of particular sample characteristics. For example, for the spectral parameter of peak intensity in an NMR spectrum, an attribute may be indicative of rotational correlation time of the molecule, exchange of hydrogen atoms with solvent, conformational dynamics of the molecule, binding of another molecule to the molecule of interest, etc. In another example, for the spectral parameter of number of peaks observed in an NMR spectrum, an attribute may be indicative of rotational correlation of the molecule, exchange of hydrogen atoms with solvent, conformational dynamics of the molecule, binding of another molecule to the molecule of interest, etc. In yet another example, for the spectral parameter of peak location in an NMR spectrum, an attribute may be indicative of conformational dynamics of the molecule, binding of another molecule to the molecule of interest, structural properties of the molecule, etc. In another example, for the spectral parameter of number of peaks observed in a spectrum acquired by mass spectrometry, an attribute may be indicative of a fragmentation pattern for the molecule of interest determined by the structure of the molecule, the reaction of the molecule with cleavage agents, for example, radiolytic agents, chemicals or enzymes, etc. In a further example, for the spectral parameter of peak location in a spectrum acquired by mass spectrometry, an attribute may be indicative of a modification to the molecule of interest, for example, modification by enzymatic reactions, modification by covalent addition, post-translation modification of a protein, etc. In yet another example, for the spectral parameter of peak location in infrared or RAMAN spectroscopy, an attribute may be indicative of structural properties of the molecule, binding of another molecule to the molecule of interest, conformational dynamics of the molecule, solvent-molecule interactions, etc.

The term “binding” refers to an association, which may be a stable association, between two molecules, e.g., between a polypeptide and a binding partner, due to, for example, electrostatic, hydrophobic, ionic and/or hydrogen-bond interactions.

The term “category” refers to a group containing at least two or more spectra comprising similar attributes for one or more spectral parameters.

The term “complex” refers to an association between at least two moieties (e.g. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand and the like. “Member of a complex” refers to one moiety of the complex, such as an antigen or ligand. “Protein complex” or “polypeptide complex” refers to a complex comprising at least one polypeptide.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

The term “domain”, when used in connection with a polypeptide, refers to a specific region within such polypeptide that comprises a particular structure and/or mediates a particular function. In a typical case, a domain of a polypeptide is a fragment of the polypeptide. In certain instances, a domain is a structurally stable domain, as evidenced, for example, by its resistance to proteolytic cleavage detected by mass spectroscopy, or by the fact that a modulator may bind to a druggable region of the domain.

The term “druggable region”, when used in reference to a polypeptide, nucleic acid, complex and the like, refers to a region of the molecule which is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions including binding pockets and sites, enzymatic active sites, interfaces between domains of a polypeptide or complex, surface grooves or contours or surfaces of a polypeptide or complex which are capable of participating in interactions with another molecule. In certain instances, the interacting molecule is another polypeptide, which may be naturally-occurring. In other instances, the druggable region is on the surface of the molecule.

Druggable regions may be described and characterized in a number of ways. For example, a druggable region may be characterized by some or all of the amino acids that make up the region, or the backbone atoms thereof, or the side chain atoms thereof (optionally with or without the Cα atoms). Alternatively, in certain instances, the volume of a druggable region corresponds to that of a carbon based molecule of at least about 200 amu and often up to about 800 amu. In other instances, it will be appreciated that the volume of such region may correspond to a molecule of at least about 600 amu and often up to about 1600 amu or more.

Alternatively, a druggable region may be characterized by comparison to other regions on the same or other molecules. For example, the term “affinity region” refers to a druggable region on a molecule (such as a polypeptide) that is present in several other molecules, in so much as the structures of the same affinity regions are sufficiently the same so that they are expected to bind the same or related structural analogs. An example of an affinity region is an ATP-binding site of a protein kinase that is found in several protein kinases (whether or not of the same origin). The term “selectivity region” refers to a druggable region of a molecule that may not be found on other molecules, in so much as the structures of different selectivity regions are sufficiently different so that they are not expected to bind the same or related structural analogs. An exemplary selectivity region is a catalytic domain of a protein kinase that exhibits specificity for one substrate. In certain instances, a single modulator may bind to the same affinity region across a number of proteins that have a substantially similar biological function, whereas the same modulator may bind to only one selectivity region of one of those proteins.

Continuing with examples of different druggable regions, the term “undesired region” refers to a druggable region of a molecule that upon interacting with another molecule results in an undesirable affect. For example, a binding site that oxidizes the interacting molecule (such as P-450 activity) and thereby results in increased toxicity for the oxidized molecule may be deemed an “undesired region”. Other examples of potential undesired regions includes regions that upon interaction with a drug decrease the membrane permeability of the drug, increase the excretion of the drug, or increase the blood brain transport of the drug. It may be the case that, in certain circumstances, an undesired region will be no longer be deemed an undesired region because the affect of the region will be favorable, e.g., a drug intended to treat a brain condition would benefit from interacting with a region that resulted in increased blood brain transport, whereas the same region could be deemed undesirable for drugs that were not intended to be delivered to the brain.

When used in reference to a druggable region, the “selectivity” or “specificity” of a molecule such as a modulator to a druggable region may be used to describe the binding between the molecule and a region. For example, the selectivity of a modulator with respect to a region may be expressed by comparison to another modulator, using the respective values of Kd (i.e., the dissociation constants for each modulator-druggable region complex) or, in cases where a biological effect is observed below the Kd, the ratio of the respective EC50's (i.e., the concentrations that produce 50% of the maximum response for the modulator interacting with each druggable region).

A “fusion protein” or “fusion polypeptide” refers to a chimeric protein as that term is known in the art and may be constructed using methods known in the art. In many examples of fusion proteins, there are two different polypeptide sequences, and in certain cases, there may be more. The sequences may be linked in frame. A fusion protein may include a domain which is found (albeit in a different protein) in an organism which also expresses the first protein, or it may be an “interspecies”, “intergenic”, etc. fusion expressed by different kinds of organisms. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. The fusion polypeptides may be fused to the N-terminus, the C-terminus, or the N- and C-terminus of the first polypeptide. Exemplary fusion proteins include polypeptides comprising a glutathione S-transferase tag (GST-tag), histidine tag (His-tag), an immunoglobulin domain or an immunoglobulin binding domain.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “having substantially similar biological activity”, when used in reference to two polypeptides, refers to a biological activity of a first polypeptide which is substantially similar to at least one of the biological activities of a second polypeptide. A substantially similar biological activity means that the polypeptides carry out a similar function in the cell, e.g., a similar enzymatic reaction or a similar physiological process, etc. For example, two homologous proteins may have a substantially similar biological activity if they are involved in a similar enzymatic reaction, e.g., they are both kinases which catalyze phosphorylation of a substrate polypeptide, however, they may phosphorylate different regions on the same protein substrate or different substrate proteins altogether. Alternatively, two homologous proteins may also have a substantially similar biological activity if they are both involved in a similar physiological process, e.g., transcription. For example, two proteins may be transcription factors, however, they may bind to different DNA sequences or bind to different polypeptide interactors. Substantially similar biological activities may also be associated with proteins carrying out a similar structural role in the cell, for example, two membrane proteins.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The terms “label” or “labeled” refer to incorporation or attachment of a detectable marker into a molecule, such as a polypeptide. Various methods of labeling polypeptides are known in the art and may be used. Examples of labels include, but are not limited to, the isotopic equivalents of atoms such as ¹³C (the lower abundance isotope of ¹²C), ¹⁵N (the lower abundance isotope of ¹⁴N) and ²H (the lower abundance isotope of ¹H).

The term “leave one out” refers to the cross-validation procedure in which a figure of merit, such as the protocol's prediction accuracy (a fraction of correct predictions) is obtained. For example, for a given case to be predicted (within the training set), a classifier is built excluding the case in question. The predictions are made in such way for every case in the training set and the resulting cumulative accuracy is reported.

The term “liquid crystal solvent” refers to aqueous environments containing entities that induce alignment anisotropy for a population of molecules in a sample subjected to spectroscopic analysis. Such liquid crystal solvents can include entities expected to have low reactivity with the sample of interest, for example, bicelles, bacteriophage, etc.

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

The term “modulator” refers to a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

The term “motif” refers to an amino acid sequence that is commonly found in a protein of a particular structure or function. Typically, a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases may be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

The term “naturally-occurring”, as applied to an object, refers to the fact that an object may be found in nature. For example, a polypeptide or polynucleotide sequence that is present in an organism (including bacteria) that may be isolated from a source in nature and which has not been intentionally modified by man in the laboratory is naturally-occurring.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The term “polypeptide”, and the terms “protein” and “peptide” which are used interchangeably herein, refers to a polymer of amino acids. Exemplary polypeptides include gene products, naturally-occurring proteins, homologs, orthologs, paralogs, fragments, and other equivalents, variants and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually the same as or substantially similar to the corresponding positions in the reference polypeptide. Such deletions may occur at the amino-terminus or carboxy-terminus of the reference polypeptide, or alternatively both or alternatively elsewhere in the sequence. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. In certain embodiments, a fragment may comprise a druggable region, and optionally additional amino acids on one or both sides of the druggable region, which additional amino acids may number from 5, 10, 15, 20, 30, 40, 50, or up to 100 or more residues. Further, fragments can include a sub-fragment of a specific region, which sub-fragment retains the function of the region from which it is derived. In another embodiment, a fragment may have immunogenic properties.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide using standard techniques for protein purification and methods described in the Exemplification section herein. Purity of a polypeptide may be determined by a number of methods known to those of skill in the art, including for example, amino-terminal amino acid sequence analysis, gel electrophoresis, mass-spectrometry analysis, etc.

The terms “recombinant protein” or “recombinant polypeptide” refer to a polypeptide which is produced by recombinant DNA techniques. An example of such techniques includes the case when DNA encoding the expressed protein is inserted into a suitable expression vector which is in turn used to transform a host cell to produce the protein or polypeptide encoded by the DNA.

The term “regulatory sequence” is a generic term used throughout the specification to refer to polynucleotide sequences, such as initiation signals, enhancers, regulators and promoters, that are necessary or desirable to affect the expression of coding and non-coding sequences to which they are operably linked. Exemplary regulatory sequences are described in Goeddel; Gene Expression Technology: Methods in Enzymology, Academic Press, San Diego, Calif. (1990), and include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. The nature and use of such control sequences may differ depending upon the host organism. In prokaryotes, such regulatory sequences generally include promoter, ribosomal binding site, and transcription termination sequences. The term “regulatory sequence” is intended to include, at a minimum, components whose presence may influence expression, and may also include additional components whose presence is advantageous, for example, leader sequences and fusion partner sequences. In certain embodiments, transcription of a polynucleotide sequence is under the control of a promoter sequence (or other regulatory sequence) which controls the expression of the polynucleotide in a cell-type in which expression is intended. It will also be understood that the polynucleotide can be under the control of regulatory sequences which are the same or different from those sequences which control expression of the naturally-occurring form of the polynucleotide.

The term “reporter gene” refers to a nucleic acid comprising a nucleotide sequence encoding a protein that is readily detectable either by its presence or activity, including, but not limited to, luciferase, fluorescent protein (e.g., green fluorescent protein), chloramphenicol acetyl transferase, β-galactosidase, secreted placental alkaline phosphatase, β-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and preferably without the need to kill the cells for signal analysis. In certain instances, a reporter gene encodes an enzyme, which produces a change in fluorometric properties of the host cell, which is detectable by qualitative, quantitative or semiquantitative function or transcriptional activation. Exemplary enzymes include esterases, β-lactamase, phosphatases, peroxidases, proteases (tissue plasminogen activator or urokinase) and other enzymes whose function may be detected by appropriate chromogenic or fluorogenic substrates known to those skilled in the art or developed in the future.

The term “sample” refers to a composition for which a spectrum analyzed by the present disclosure is collected. Samples can include, for example, polypeptides, polynucleotides, small molecules, or different combinations of such molecules and can be in various environments, for examples, solution, liquid crystal solvent, crystalline form, etc. “Sample spectra” refer to the spectra collected on such samples.

The term “scoring” of a spectral parameter refers to assigning a rating based on the attributes of the spectral parameter. The rating can represent, for example, a quality of an attribute, a characteristic property of the sample reflected in the attribute, etc. In certain cases the scoring is in numerical form. Such scoring can be accomplished, for example, by a determination of a combination of the variables defining the attributes, determining a probability distribution for the variables defining the attributes, etc. for the attributes of one or more spectral parameters in a category of spectra. Such scoring may include using statistical relationships, for example, Bayesian classifiers, neural networks, decision trees, etc.

The term “small molecule” refers to a compound, which has a molecular weight of less than about 5 kD, less than about 2.5 kD, less than about 1.5 kD, or less than about 0.9 kD. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened by the systems and methods of the present disclosure. The term “small organic molecule” refers to a small molecule that is often identified as being an organic or medicinal compound, and does not include molecules that are exclusively nucleic acids, peptides or polypeptides.

The term “soluble” as used herein with reference to a polypeptide or other protein, means that upon expression in cell culture, at least some portion of the polypeptide or protein expressed remains in the cytoplasmic fraction of the cell and does not fractionate with the cellular debris upon lysis and centrifugation of the lysate. Solubility of a polypeptide may be increased by a variety of art recognized methods, including fusion to a heterologous amino acid sequence, deletion of amino acid residues, amino acid substitution (e.g., enriching the sequence with amino acid residues having hydrophilic side chains), and chemical modification (e.g., addition of hydrophilic groups). The solubility of polypeptides may be measured using a variety of art recognized techniques, including, dynamic light scattering to determine aggregation state, UV absorption, centrifugation to separate aggregated from non-aggregated material, and SDS gel electrophoresis (e.g., the amount of protein in the soluble fraction is compared to the amount of protein in the soluble and insoluble fractions combined). When expressed in a host cell, polypeptides may be at least about 1%, 2%, 5%, 10%, 20%, 30%, 40%, 50%, 60%, 70%, 80%, 90% or more soluble, e.g., at least about 1%, 2%, 5%, 10%, 20%, 30%, 40%, 50%, 60%, 70%, 80%, 90% or more of the total amount of protein expressed in the cell is found in the cytoplasmic fraction. In certain embodiments, a one liter culture of cells expressing a polypeptide will produce at least about 0.1, 0.2, 0.5, 1, 2, 5, 10, 20, 30, 40, 50 milligrams or more of soluble protein. In an exemplary embodiment, a polypeptide is at least about 10% soluble and will produce at least about 1 milligram of protein from a one liter cell culture.

The term “specifically hybridizes” refers to detectable and specific nucleic acid binding. Polynucleotides, oligonucleotides and nucleic acids selectively hybridize to nucleic acid strands under hybridization and wash conditions that minimize appreciable amounts of detectable binding to nonspecific nucleic acids. High stringency conditions may be used to achieve selective hybridization conditions as known in the art and discussed herein. Generally, the nucleic acid sequence homology between the polynucleotides, oligonucleotides, and nucleic acids and a nucleic acid sequence of interest will be at least 30%, 40%, 50%, 60%, 70%, 80%, 85%, 90%, 95%, 98%, 99%, or more. In certain instances, hybridization and washing conditions are performed at high stringency according to conventional hybridization procedures.

The term “spectrum” refers to the distribution of a characteristic or characteristics of a physical system or phenomenon. Such spectra may be acquired as a result of a variety of techniques intended to measure such characteristics of a physical system including, for example, NMR spectroscopy, mass spectrometry, infrared and RAMAN spectroscopy, chromatography, etc. The measured characteristics of a physical system will vary depending on the processes used, for example, in the case of NMR spectroscopy, such characteristics may include alignment of spins for nuclei in a magnetic field; in the case of mass spectrometry such characteristics may include molecular mass and charge; in the case of infrared and RAMAN Spectroscopy such characteristics may include absorption of light of a particular wavelength, etc.

The term “spectral parameter” refers to a feature in a spectrum characteristic of a sample. Spectral parameters will vary with the type of technique used to analyze the sample. For example, in the case of spectra collected by the technique of NMR spectroscopy, such spectral parameters may include, for example, peak intensity, peak location, the number of peaks observed versus the number expected, peak shape, etc. As another example, for spectra collected by the technique of mass spectrometry, such spectral parameters may include, for example, peak location, number of peaks observed etc. In yet another example, for spectra collected by the technique of infrared and RAMAN spectroscopy, such spectral parameters may include, for example, peak intensity, peak location, number of peaks observed. The units used to measure said spectral parameters will also vary with the type of technique used. For example, in the case of spectra collected by the technique of NMR spectroscopy, the spectral parameter of peak location may be measured by, for example, chemical shift, frequency of magnetic resonance, etc. In another example, in the case of spectra collected by the technique of mass spectrometry, the spectral parameter of peak location may be measured by, for example, molecular mass, charge, etc. In yet another example, in the case of spectra collected by the technique of infrared and RAMAN spectroscopy, the spectral parameter peak location may be measured by units of wavelength, wave number of vibration or rotation between two or more atoms, or sets of atoms within a sample.

The term “structural motif”, when used in reference to a polypeptide, refers to a polypeptide that, although it may have different amino acid sequences, may result in a similar structure, wherein by structure is meant that the motif forms generally the same tertiary structure, or that certain amino acid residues within the motif, or alternatively their backbone or side chains (which may or may not include the Cα atoms of the side chains) are positioned in a like relationship with respect to one another in the motif.

The term “test compound” refers to a molecule to be tested by systems and methods of the present disclosure as a putative modulator of one or more molecules of interest or other biological entity or process. A test compound is usually not known to bind to the molecules of interest. The term “control test compound” refers to a compound known to bind to a molecule of interest (e.g., a known agonist, antagonist, partial agonist or inverse agonist). The term “test compound” does not include a chemical added as a control condition that alters the function of the molecule to determine signal specificity in an assay. Such control chemicals or conditions include chemicals that 1) nonspecifically or substantially disrupt protein structure (e.g., denaturing agents (e.g., urea or guanidinium), chaotropic agents, sulfhydryl reagents (e.g., dithiothreitol and β-mercaptoethanol), and proteases), 2) generally inhibit cell metabolism (e.g., mitochondrial uncouplers) and 3) non-specifically disrupt electrostatic or hydrophobic interactions of a protein (e.g., high salt concentrations, or detergents at concentrations sufficient to non-specifically disrupt hydrophobic interactions). Further, the term “test compound” also does not include compounds known to be unsuitable for a therapeutic use for a particular indication due to toxicity of the subject. In certain embodiments, various predetermined concentrations of test compounds are used for screening such as 0.01 μM, 0.1 μM, 1.0 μM, and 10.0 μM. Examples of test compounds include, but are not limited to, peptides, nucleic acids, carbohydrates, and small molecules. The term “novel test compound” refers to a test compound that is not in existence as of the filing date of this application. In certain assays using novel test compounds, the novel test compounds comprise at least about 50%, 75%, 85%, 90%, 95% or more of the test compounds used in the assay or in any particular trial of the assay.

The term “training set” refers to one or more spectra that are associated with categories by the present disclosure. The spectra of a training set may be obtained on one molecule in a plurality of environments, a molecule in combination with different molecule(s) in the same or different environments, or a plurality of different molecules (for example, proteins, nucleic acids or small molecules). The spectra of said training set may be acquired by a variety of techniques, for example, NMR spectroscopy, mass spectrometry, infrared and RAMAN spectroscopy, chromatography, etc.

The term “vector” refers to a nucleic acid capable of transporting another nucleic acid to which it has been linked. One type of vector which may be used in accord with the disclosure is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules which, in their vector form are not bound to the chromosome. In the present specification, “plasmid” and “vector” are used interchangeably as the plasmid is the most commonly used form of vector. Such other forms of expression vectors which serve equivalent functions and which become known in the art subsequently hereto can be used to produce proteins or fragments thereof for which spectra evaluated by the systems and methods of the present disclosure are acquired.

2. Evaluation of Spectra

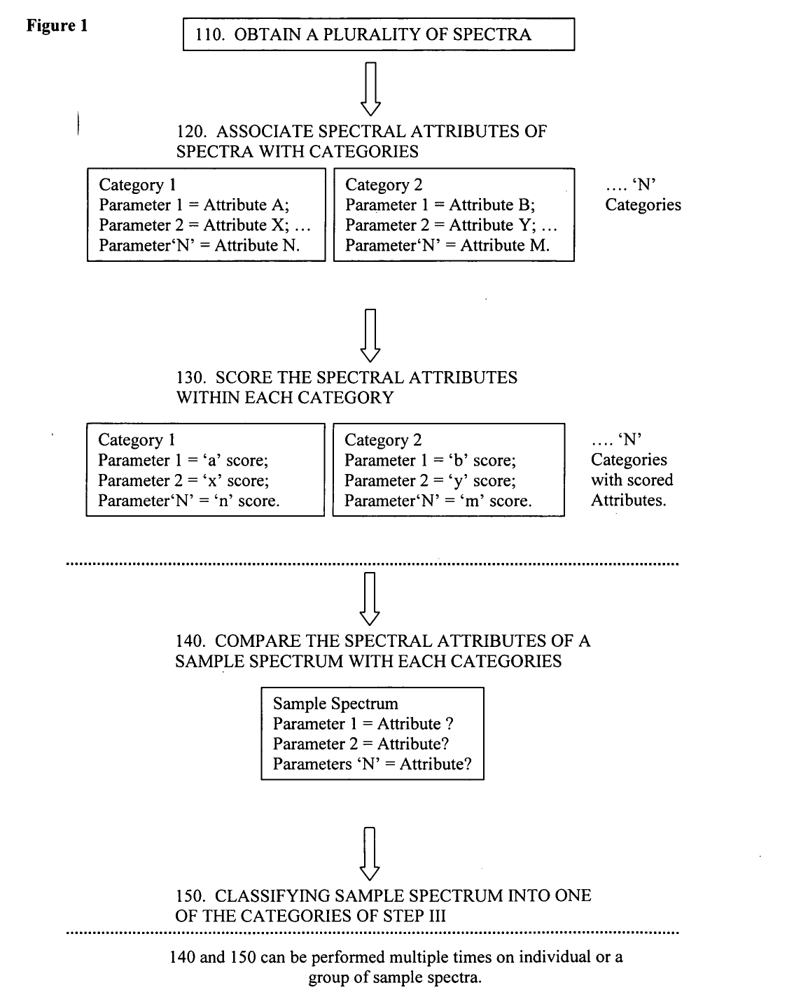

Embodiments of the present disclosure include systems and methods for the evaluation of spectra. Certain embodiments of the present disclosure are represented by the whole or parts of the schematic of FIG. 1. In certain embodiments, a training set containing a plurality of spectra is obtained (110). A training set of spectra can be collected by a variety of techniques including, for example, NMR spectroscopy, mass spectrometry, infrared and RAMAN spectroscopy, chromatography, etc. on samples comprising proteins, nucleic acids and small molecules. The spectra obtained may be one, two or multidimensional.

The spectra can then be categorized based on the attributes of at least two or more spectral parameters (120). The attributes of a spectral parameter will differ depending on the sample(s) and the technique for which the spectra are collected.

In the case of NMR spectra, the spectral parameters of peak location can be measured in units of chemical shift. A particular chemical shift or a range of chemical shifts may be chosen to reflect a property of the sample. For example, for a protein, a certain chemical shift or range of chemical shifts may be indicative of secondary or tertiary structure for a folded protein. In another example, the spectral parameter of fraction of peaks observed may be a ratio of observed peaks to expected peaks for a particular sample. Such a fraction of peaks observed may be indicative of secondary or tertiary structure for a folded protein. In yet another example, the spectral parameters of peak intensity may be the width of one or more peaks in a spectrum. The width of a peak may be indicative of the rotational correlation time for a sample.

In certain embodiments, after the two or more spectra are associated with different categories based on their attributes, the spectral parameters of the two or more spectra within the categories are scored (130). The scoring can be accomplished by, for example, a determination of a combination of the variables defining the attributes, determining a probability distribution for the variables defining the attributes, etc. for the attributes of one or more spectral parameters in a category of spectra. Such scoring may include using statistical relationships, for example, Bayesian classifiers, neural networks, decision trees, etc. The attributes observed may be statistically correlated for one or more spectral parameters in a category of spectra. The scoring may be accomplished using one or more processors.

A number of statistical methods and algorithms can be used to achieve the scoring of 130. In certain embodiments of the disclosure Bayesian classifiers can be used for scoring. In one example of such an embodiment, naïve Bayes classifiers can be used which assume statistical independence of the attributes being evaluated by the systems and methods of the present disclosure. In another example of such an embodiment, Bayesian networks can be used which learn a multidimensional probability distribution in the attributes/classes space. In a particular embodiment neural networks can be used for the scoring of 130. In other embodiments, scoring can be achieved by using decision trees. The number of spectra required for an accuracy in scoring will depend upon the statistical approach applied. For example, more training set spectra could be required when using Bayesian networks in order to establish a multidimensional probability distribution than for a naïve Bayes approach.

Other related approaches such as a “jack-knifing” procedure (A Bayesian System Integrating Expression Data with Sequence Patterns for Localizing Proteins: Comprehensive Application to the Yeast Genome, Amar Drawid and Mark Gerstein, J. Mol. Biol. (2000) 301, 1059-1075), or advanced attribute search algorithms (stochastic, genetic algorithms, etc) are readily applicable within this strategy.

In certain embodiments, one or more sample spectra are collected and the attributes of the spectral parameters are determined for each sample spectrum. The attributes of the spectral parameters of the sample spectra are compared with those of the spectral parameters of the categories of 130 (140). Based on the comparison the sample spectra are classified into one of the categories of 130 (150). In certain embodiments, 140 and 150 can be accomplished on sample spectrum one spectrum at a time. In further embodiments 140 and 150 can be accomplished simultaneously for multiple sample spectra.

The systems and methods of the present disclosure are not limited to a particular hardware or software configuration, and may find applicability in many computing or processing environments. The systems and methods can be implemented in hardware or software, or a combination of hardware and software. The systems and methods of the present disclosure and the techniques and processes used to acquire spectra for evaluation by the present disclosure can be implemented in one or more computer programs, where a computer program can be understood to include one or more processor executable instructions. The computer program(s) can execute on one or more programmable processors, and can be stored on one or more storage media readable by the processor (including volatile and non-volatile memory and/or storage elements), one or more input devices, and/or one or more output devices. The processor thus can access one or more input devices to obtain input data, and can access one or more output devices to communicate output data. The input and/or output devices can include one or more of the following: Random Access Memory (RAM), Redundant Array of Independent Disks (RAID), floppy drive, CD, DVD, magnetic disk, internal hard drive, external hard drive, memory stick, or other storage device capable of being accessed by a processor as provided herein, where such aforementioned examples are not exhaustive, and are for illustration and not limitation.

Certain embodiments of the present disclosure also include computer products for implementing the evaluation of spectra. Such computer products can be implemented using one or more high level procedural or object-oriented programming languages to communicate with a computer system; however, the programs can be implemented in assembly or machine language, if desired. The language can be compiled or interpreted.

The processor(s) used to implement the evaluation of spectra embodied by the systems and methods of the present disclosure can be embedded in one or more devices that can be operated independently or together in a networked environment, where the network can include, for example, a Local Area Network (LAN), wide area network (WAN), and/or can include an intranet and/or the internet and/or another network. The network(s) can be wired or wireless or a combination thereof and can use one or more communications protocols to facilitate communications between the different processors. The processors can be configured for distributed processing and can utilize, in some embodiments, a client-server model as needed. Accordingly, the techniques and processes can utilize multiple processors and/or processor devices, and the processor instructions can be divided amongst such single or multiple processor/devices.

The device(s) or computer systems that integrate with the processor(s) can include, for example, a personal computer(s), workstation (e.g., Sun, HP), personal digital assistant (PDA), handheld device such as cellular telephone, laptop, handheld, or another device capable of being integrated with a processor(s) that can operate as provided herein.

References to “a processor” or “the processor” can be understood to include one or more processors that can communicate in a stand-alone and/or a distributed environment(s), and thus can be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network using a variety of communications protocols, and unless otherwise specified, can be arranged to include a combination of external and internal memory devices, where such memory can be contiguous and/or partitioned based on the application. Accordingly, references to a database can be understood to include one or more memory associations, where such references can include commercially available database products (e.g., SQL, Informix, Oracle) and also proprietary databases, and may also include other structures for associating memory such as links, queues, graphs, trees, with such structures provided for illustration and not limitation.

3. Applications of Spectral Evaluation

One aspect of the disclosure pertains to the evaluation of spectra acquired by spectroscopic techniques. Such techniques can include for example, NMR spectroscopy, mass spectrometry, infrared and RAMAN spectroscopy, chromatography, etc. In certain embodiments of the exemplary applications described below, a training set of spectra are evaluated as described above and in the schematic of FIG. 1. In further embodiments, sample spectra are collected and classified into one of the categories of 130, as described above. The classification of spectra results in the identification of particular sample characteristics which will be detailed in the exemplary applications that follow.

(i) Evaluation of Nuclear Magnetic Resonance (NMR) Spectra

In one embodiment, the present disclosure contemplates evaluating spectra acquired by the technique of NMR spectroscopy.

In certain embodiments, the system and methods of the present disclosure can be used to identify samples with desirable spectroscopic properties for structural analysis by NMR. In such an embodiment, purified molecules can be made and subjected to NMR spectroscopic analysis, thereby acquiring spectra appropriate for evaluation. Such a method can comprise, (a) generating a purified molecule of interest, for example, a protein, nucleic acid, or small molecule, or a fragment thereof; (b) preparing a sample of the molecule in an appropriate solution, liquid crystal solvent or crystalline form; (c) subjecting the sample to NMR spectroscopic analysis, and (d) repeating (a) through (c) for a variety of molecules, thereby acquiring a plurality of spectra for evaluation by the present disclosure. In certain embodiments, a training set of spectra collected on a plurality of molecules is evaluated using attributes of spectral parameters indicative of desirable spectroscopic properties by systems and methods of the present disclosure. In further embodiments, sample spectra are classified using attributes of spectral parameters indicative of desirable spectroscopic properties by systems and methods of the present disclosure thereby resulting in identification of samples with said spectroscopic properties.

In another embodiment, the present disclosure contemplates evaluating spectra acquired by the technique of NMR spectroscopy for the purpose of determining solution or liquid crystal solvent conditions appropriate for NMR analysis of a molecule or molecules. Such a method can comprise, (a) generating a purified molecule of interest, for example, a protein, nucleic acid, or small molecule, or a fragment thereof; (b) preparing a sample of the molecule in an appropriate solution or liquid crystal solvent condition; (c) subjecting the sample to NMR spectroscopic analysis, and (d) repeating (a) through (c) for a variety of solutions or liquid crystal solvent conditions, thereby acquiring a plurality of spectra for evaluation by the present disclosure. In certain embodiments, a training set of spectra collected on a plurality of molecules is evaluated using attributes of spectral parameters indicative of desirable spectroscopic properties associated with solution or liquid crystal solvent conditions. In further embodiments, sample spectra are classified using attributes of spectral parameters indicative of desirable spectroscopic properties of said conditions thereby resulting in identification of conditions with said spectroscopic properties.

In certain embodiments, one can use the system and methods of the present disclosure in order to monitor the interaction between a selected molecule, for example, a protein, nucleic acid, or small molecule and one or more molecules by evaluating spectra acquired by the technique of NMR spectroscopy. In such an embodiment, the disclosure can include detecting, designing and characterizing interactions between a selected molecule and test molecules, including proteins, nucleic acids and small molecules, utilizing NMR techniques. In one such embodiment, the present disclosure contemplates evaluating spectra for identifying test molecules that bind to a molecule of interest (for example, a protein, nucleic acid or small molecule or a fragment thereof). Such a method can comprise: (a) generating a first NMR spectrum of the molecule, or a fragment thereof; (b) exposing the molecule to one or more test molecules; (c) generating a second NMR spectrum of the molecule which has been exposed to one or more test molecules; and (d) repeating (a) through (c) for a plurality of test molecules, thereby acquiring spectra for evaluation by the present disclosure wherein differences between spectra are indicative of test molecules that have bound to the molecule of interest. In certain embodiments, a training set of spectra collected on a plurality of molecules bound to the molecule of interest is evaluated using attributes of spectral parameters indicative of desirable spectroscopic properties of the binding of said molecules. In further embodiments, sample spectra are classified using attributes of spectral parameters indicative of said desirable spectroscopic properties thereby resulting in identification of molecules that bind the molecule of interest.

In another such embodiment, the present disclosure contemplates evaluating spectra obtained on a plurality of conditions while a molecule is in a complex with another molecule. Such a method can comprise: (a) generating a purified molecule of interest, for example, a protein, nucleic acid or small molecule, or a fragment thereof; (b) forming a complex between the molecule and the test molecule; (c) subjecting the complex to NMR spectroscopic analysis, and (d) repeating (a) through (c) for a variety of conditions, thereby acquiring a plurality of spectra for evaluation by the methods of the present disclosure. In certain embodiments, a training set of spectra collected on a plurality of conditions is evaluated using attributes of spectral parameters indicative of desirable spectroscopic properties of complex formation between said molecules. In further embodiments, sample spectra are classified using attributes of spectral parameters indicative of said desirable spectroscopic properties thereby resulting in identification of said desirable conditions.

Briefly, the acquisition of NMR spectra for evaluation by the systems and methods of the present disclosure involves, for example, placing the material to be examined in a powerful magnetic field and irradiating it with radio frequency (rf) electromagnetic radiation. The nuclei of the various atoms will align themselves with the magnetic field until energized by the rf radiation. The nuclei absorb this resonant energy which can be detected at a frequency dependent on i) the type of nucleus and ii) its atomic environment. Moreover, resonant energy may be passed from one nucleus to another, either through bonds or through three-dimensional space, thus giving information about the environment of a particular nucleus and nuclei in its vicinity.

However, it is important to recognize that not all nuclei are NMR active. Indeed, not all isotopes of the same element are active. For example, whereas “ordinary” hydrogen, ¹H, is NMR active, heavy hydrogen (deuterium), ²H, is not active in the same way because it does not resonate at the same frequency. Thus, any material that normally contains ¹H hydrogen may be rendered “invisible” in the hydrogen NMR spectrum by replacing all or almost all the ¹H hydrogens with ²H. It is for this reason that NMR spectroscopic analyses of water-soluble materials frequently are performed in ²H₂O (or deuterium) to eliminate the water signal.

Conversely, “ordinary” carbon, ¹²C, is NMR inactive whereas the stable isotope, ¹³C, present to about 1% of total carbon in nature, is active. Similarly, while “ordinary” nitrogen, ¹⁴N, is NMR active, it has undesirable properties for NMR and resonates at a different frequency from the stable isotope ¹⁵N, present to about 0.4% of total nitrogen in nature.

By labeling molecules with ¹⁵N and ¹⁵N/¹³C, it is possible to conduct analytical NMR of macromolecules with weights of up to 15 kD and 40 kD, respectively. More recently, partial deuteration of the protein in addition to ¹³C- and ¹⁵N-labeling has increased the possible weight of proteins and protein complexes for NMR analysis still further, to approximately 60-70 kD. See Shan et al., J. Am. Chem. Soc., 118:6570-6579 (1996); L. E. Kay, Methods Enzymol., 339:174-203 (2001); and K. H. Gardner & L. E. Kay, Annu Rev Biophys Biomol Struct., 27:357-406 (1998); and references cited therein.

Isotopic substitution may be accomplished by growing a bacterium or yeast or other type of cultured cells, transformed by genetic engineering to produce the protein of choice, in a growth medium containing ¹³C-, ¹⁵N- and/or ²H-labeled substrates. In certain instances, bacterial growth media consists of ¹³C-labeled glucose and/or ¹⁵N-labeled ammonium salts dissolved in D₂O where necessary. Kay, L. et al., Science, 249:411 (1990) and references therein and Bax, A., J. Am. Chem. Soc., 115, 4369 (1993). More recently, isotopically labeled media especially adapted for the labeling of bacterially produced macromolecules have been described. See U.S. Pat. No. 5,324,658.

The goal of these methods has been to achieve universal and/or random isotopic enrichment of all of the amino acids of the protein. By contrast, other methods allow only certain residues to be relatively enriched in ¹H, ²H, ¹³C and ¹⁵N. See Kay et al., J. Mol. Biol., 263, 627-636 (1996) and Kay et al., J. Am. Chem. Soc., 119, 7599-7600 (1997) have described methods whereby isoleucine, alanine, valine and leucine residues in a protein may be labeled with ²H, ¹³C and ¹⁵N, and may be specifically labeled with ¹H at the terminal methyl position. In this way, study of the proton-proton interactions between some amino acids may be facilitated. Similarly, a cell-free system may be used wherein a transcription-translation system derived from E. coli was used to express human Ha-Ras protein incorporating ¹⁵N into serine and/or aspartic acid. Techniques for producing isotopically labeled proteins and macromolecules, such as glycoproteins, in mammalian or insect cells have been described. See U.S. Pat. Nos. 5,393,669 and 5,627,044; Weller, C. T., Biochem., 35, 8815-23 (1996) and Lustbader, J. W., J. Biomol. NMR, 7, 295-304 (1996). Other methods for producing polypeptides and other molecules with labels appropriate for NMR are known in the art.

The present disclosure contemplates using a variety of solvents which are appropriate for NMR. For ¹H NMR, a deuterium lock solvent may be used. Exemplary deuterium lock solvents include acetone (CD₃COCD₃), chloroform (CDCl₃), dichloro methane (CD₂Cl₂), methylnitrile (CD₃CN), benzene (C₆D₆), water (D₂O), diethylether ((CD₃CD₂)₂O), dimethylether ((CD₃)₂O), N,N-dimethylformamide ((CD₃)₂NCDO), dimethyl sulfoxide (CD₃SOCD₃), ethanol (CD₃CD₂OD), methanol (CD₃OD), tetrahydrofuran (C₄D₈O), toluene (C₆D₅CD₃), pyridine (C₅D₅N) and cyclohexane (C₆H₁₂). For example, the present disclosure contemplates a composition comprising polypeptide, polynucleotides or small molecules and a deuterium lock solvent. In a particular example, for ¹⁵N-HSQCs of proteins the solvent is water with only 5-10% deuterium lock in the form of D₂O.

In certain embodiments, one can use the systems and methods of the present disclosure in order to identify protein samples with desirable spectroscopic properties for structural analysis by NMR. In such an embodiment, purified proteins can be made with naturally occurring percentages of NMR active isotopes or isotopically enriched by the methods described above and subjected to NMR spectroscopic analysis, thereby acquiring spectra appropriate for evaluation. Such an embodiment can comprise, (a) generating a purified protein of interest, for example, a protein from an endogenous source or a fragment thereof, a protein expressed in bacteria, yeast, mammalian, or other cells containing an appropriate expression system or a fragment thereof, a synthetically made protein or a fragment thereof, an isotopically enriched protein or a fragment thereof, etc.; (b) preparing a sample of the protein in an appropriate solution, liquid crystal solvent or crystalline form; (c) subjecting the sample to NMR spectroscopic analysis, and (d) repeating (a) through (c) for a variety of proteins, thereby acquiring a plurality of spectra for evaluation by the present disclosure. In certain embodiments, a training set of spectra collected on a plurality of proteins is evaluated using attributes of spectral parameters indicative of desirable spectroscopic properties by systems and methods of the present disclosure. In further embodiments, sample spectra are classified using attributes of spectral parameters indicative of desirable spectroscopic properties by systems and methods of the present disclosure thereby resulting in identification of proteins with said spectroscopic properties.

In further embodiments, one can use the systems and methods of the present disclosure in order to identify sample conditions which result in desirable spectroscopic properties for one or more proteins for structural analysis by NMR. Such an embodiment can comprise, (a) generating a purified protein of interest, for example, a protein from an endogenous source or a fragment thereof, a protein expressed in bacteria, yeast, mammalian, or other cells containing an appropriate expression system or a fragment thereof, a synthetically made protein or a fragment thereof, an isotopically enriched protein or a fragment thereof, etc.; (b) preparing a sample of the protein in an appropriate solution or liquid crystal solvent; (c) subjecting the sample to NMR spectroscopic analysis, and (d) repeating (a) through (c) for a variety of solutions or liquid crystal solvents, thereby acquiring a plurality of spectra for evaluation by the present disclosure. In certain embodiments, a training set of spectra collected on a plurality of conditions is evaluated using attributes of spectral parameters indicative of desirable spectroscopic properties. In further embodiments, sample spectra are classified using attributes of spectral parameters indicative of desirable spectroscopic properties thereby resulting in identification of conditions that result in said spectroscopic properties.

In certain embodiments of the present disclosure, the interaction between a selected protein and one or more molecules can also be monitored by evaluating spectra acquired by the technique of NMR spectroscopy. In such an embodiment, the disclosure can include detecting, designing and characterizing interactions between a selected protein and test molecules, including proteins, nucleic acids and small molecules, utilizing NMR techniques. In such an embodiment, purified proteins can be made with naturally occurring percentages of NMR active isotopes or isotopically enriched by the methods described above and subjected to NMR spectroscopic analysis, thereby acquiring spectra appropriate for evaluation. Such a method can comprise: (a) generating a purified protein of interest, for example, a protein from an endogenous source or a fragment thereof, a protein expressed in bacteria, yeast, mammalian, or other cells containing an appropriate expression system or a fragment thereof, a synthetically made protein or a fragment thereof, an isotopically enriched protein or a fragment thereof, etc.; (b) forming a complex between the protein and one or more test molecules; (c) subjecting the complex to NMR spectroscopic analysis, and (d) repeating (a) through (c) for a variety of molecules, thereby acquiring a plurality of spectra for evaluation by the methods of the present disclosure. In certain embodiments, a training set of spectra collected on the protein of interest in complex with a plurality of test molecules is evaluated using attributes of spectral parameters indicative of desirable spectroscopic properties associated with complex formation. In further embodiments, sample spectra are classified using attributes of spectral parameters indicative of desirable spectroscopic properties of said complex formation thereby resulting in identification of molecules that bind the protein of interest.

In further embodiments the present disclosure contemplates evaluating spectra obtained on a plurality of conditions for one or more molecules in complex with a protein of interest. Such a method can comprise: (a) generating a purified protein of interest, for example, a protein from an endogenous source or a fragment thereof, a protein expressed in bacteria, yeast, mammalian, or other cells containing an appropriate expression system or a fragment thereof, a synthetically made protein or a fragment thereof, an isotopically enriched protein or a fragment thereof, etc.; (b) forming a complex between the protein and one or more test molecules in an appropriate solution or liquid crystal solvent condition; (c) subjecting the complex to NMR spectroscopic analysis, and (d) repeating (a) through (c) for a variety of solution or liquid crystal solvent conditions, thereby acquiring a plurality of spectra for evaluation by the methods of the present disclosure. In certain embodiments, a training set of spectra collected on a plurality of proteins in complex with one or more molecules is evaluated using attributes of spectral parameters indicative of desirable spectroscopic properties associated with conditions appropriate for complex formation. In further embodiments, sample spectra are classified using attributes of spectral parameters indicative of desirable spectroscopic properties thereby resulting in identification of desirable conditions.

In certain embodiments the protein spectra evaluated by the present disclosure can include 2-dimensional ¹H-¹⁵N HSQC (Heteronuclear Single Quantum Coherence) spectra. The 2-dimensional ¹H-¹⁵N HSQC spectrum provides a diagnostic fingerprint of conformational state, aggregation level, state of protein folding, and dynamic properties of a polypeptide (Yee et al, PNAS 99, 1825-30 (2002)). Polypeptides in aqueous solution usually populate an ensemble of 3-dimensional structures which can be determined by NMR. When the polypeptide is a stable globular protein or domain of a protein, then the ensemble of solution structures is one of very closely related conformations. In this case, one peak is expected for each non-proline residue with a dispersion of resonance frequencies with roughly equal intensity. Additional pairs of peaks from side-chain NH₂ groups are also often observed, and correspond to the approximate number of Gln and Asn residues in the protein. This type of HSQC spectra usually indicates that the protein is amenable to structure determination by NMR methods. Methods of the present disclosure can be applied to evaluated such HSQC spectra obtained for a plurality of proteins, a protein in a plurality of solution, liquid crystal solvent or crystalline conditions or one or more proteins in complex with one or more molecules in order to determine samples or conditions appropriate for 3D structural determination by NMR, binding of the protein to other molecules, the spectroscopic properties of individual proteins etc.

In certain embodiments of the present disclosure, the attributes of a spectral parameter, for example, number of peaks observed, peak intensity, peak location, etc. can be indicative of the spectroscopic properties of the protein(s) for which HSQC spectra are evaluated. For example, if the HSQC spectrum shows well-dispersed peaks (a number of peak locations) but there are either too few or too many in number, and/or the peak intensities differ throughout the spectrum, then the protein likely does not exist in a single globular conformation. Such spectral features are indicative of conformational heterogeneity with slow or nonexistent inter-conversion between states (more than the expected number of peaks observed) or the presence of dynamic processes on an intermediate timescale that can broaden and obscure the NMR signals. Proteins with this type of spectrum can sometimes be stabilized into a single conformation by changing either the protein construct, the solution conditions, temperature or by binding of another molecule. Embodiments of the present disclosure can be used to screen for such desired protein constructs, solution, liquid crystal solvent conditions, temperature, binding of molecules etc.

In certain embodiments of the present disclosure, the spectral attribute of peak location can be indicative of the spectroscopic properties of the protein(s) for which HSQC spectra are evaluated. Proteins that are largely unfolded, e.g., having very little regular secondary structure, result in ¹H-¹⁵N H SQC spectra in which the peaks are all very narrow and intense, but have very little spectral dispersion in the ¹⁵N-dimension. This reflects the fact that many or most of the amide groups of amino acids in unfolded polypeptides are solvent exposed and experience similar chemical environments resulting in similar ¹H chemical shifts.

The evaluation of multiple sample ¹H-¹⁵N HSQC by the systems and methods of the present disclosure can thus allow the rapid characterization of the conformational state, aggregation level, state of protein folding, and dynamic properties of a plurality of polypeptides. Additionally, other 2D spectra such as ¹H-¹³C HSQC, or HNCO spectra can also be used in a similar manner by the evaluation processes of the present disclosure.

NMR spectra acquired for a polypeptide in the presence and absence of a plurality of test compounds (e.g., a polypeptide, nucleic acid or small molecule) may be used to characterize interactions between a polypeptide and another molecule using the systems and methods of the present disclosure. Because the ¹H-¹⁵N HSQC spectrum and other simple 2D NMR experiments can be obtained very quickly (on the order of minutes depending on protein concentration and NMR instrumentation), they are very useful for rapidly testing whether a polypeptide is able to bind to another molecule.

In certain embodiments of the present disclosure, the attributes of the spectral parameter of peak location can be defined as changes in the resonance frequency (in one or both dimensions) of one or more peaks in the HSQC spectrum and can be indicative of an interaction with another molecule. Often only a subset of the peaks will have changes in resonance frequency upon binding to another molecule, allowing one to select for test compounds that interact with certain residues directly involved in the interaction or involved in conformational changes as a result of the interaction. In certain embodiments of the present disclosure, the spectral parameter of peak intensity can be used to evaluate the spectra acquired on a protein of interest interacting with test molecules. In such an embodiment, if the interacting molecule is relatively large (protein or nucleic acid) the peak intensity will decrease or disappear entirely due to the increased rotational correlation time of the complex or due to intermediate exchange on the NMR timescale (i.e., exchanging on and off the polypeptide at a frequency that is similar to the difference in resonance frequency of the monitored nuclei in the liganded and unliganded states).

To facilitate the acquisition of NMR data on a large number of compounds (e.g., a library of synthetic or naturally-occurring small organic compounds), a sample changer may be employed. Using the sample changer, a larger number of samples, numbering 60 or more, may be run unattended. To facilitate processing of the NMR data, computer programs are used to transfer and automatically process the multiple one-dimensional and two-dimensional NMR data.

A ¹⁵N- or ¹³C-labeled polypeptide can be exposed to a number of molecules present in a library of compounds such as a plurality of small molecules. Such molecules are typically dissolved in perdeuterated dimethylsulfoxide. The compounds in the library may be purchased from vendors or created according to desired needs.

The NMR screening process of the present disclosure can utilize a range of test compound concentrations, e.g., from about 0.05 to about 1.0 mM. At those exemplary concentrations, compounds which are acidic or basic may significantly change the pH of buffered protein solutions. Chemical shifts are sensitive to pH changes as well as direct binding interactions, and false-positive chemical shift changes, which are not the result of test compound binding but of changes in pH, may therefore be observed. It may therefore be necessary to ensure that the pH of the buffered solution does not change upon addition of the test compound.

(ii) Evaluation of Mass Spectrometry Spectra

In one embodiment, the present disclosure contemplates evaluating spectra acquired by the technique of mass spectroscopy.

In certain embodiments, the present disclosure contemplates evaluating spectra collected by mass spectrometry for the purpose of screening molecules. Such an embodiment can comprise, (a) generating a purified molecule of interest, for example, a protein, nucleic acid, or small molecule, or fragment thereof; (b) preparing a sample of the molecule in an appropriate solvent; (c) subjecting the sample to mass spectrometry, and (d) repeating (a) through (c) for a variety of molecules, thereby generating a plurality of spectra for evaluation by the methods of the present disclosure. In certain embodiments, a training set of spectra collected on a plurality of molecules is evaluated using attributes of spectral parameters indicative of desirable properties of the molecules. In further embodiments, sample spectra are classified using attributes of spectral parameters indicative of properties of said molecules thereby resulting in identification of samples by their properties.

In further embodiments, the present disclosure contemplates evaluating spectra collected by mass spectrometry for the purpose of identifying modifications (for example, modification by enzymatic reactions, modification by covalent addition, post-translational modifications e.g., phosphorylation) etc. of a molecule. Such an embodiment can comprise, (a) generating a purified molecule of interest, for example, a protein, nucleic acid, or small molecule, or fragment thereof; (b) preparing a sample of the molecule in an appropriate solvent; (c) subjecting the sample to mass spectrometry, and (d) repeating (a) through (c) for a variety of molecules, the same molecule generated by a variety of reactions, or the same molecule purified from a variety of sources, thereby generating a plurality of spectra for evaluation by the methods of the present disclosure. In certain embodiments, a training set of spectra collected on a plurality of molecules is evaluated using attributes of spectral parameters indicative of spectroscopic properties associated with the modifications. In further embodiments, sample spectra are classified using attributes of spectral parameters indicative of spectroscopic properties thereby resulting in identification of samples by their modifications.

Typically, spectra acquired by the technique of mass spectroscopy first requires isolation of the molecule. In certain embodiments of the present disclosure, spectra are acquired by mass spectrometry after the molecule is subjected to either chemical or enzymatic reactions.

Various mass spectrometers may be used within the present disclosure. Representative examples include: triple quadrupole mass spectrometers, magnetic sector instruments (magnetic tandem mass spectrometer, JEOL, Peabody, Mass.), ionspray mass spectrometers (Bruins et al., Anal Chem. 59:2642-2647, 1987), electrospray mass spectrometers (including tandem, nano- and nano-electrospray tandem) (Fenn et al., Science 246:64-71, 1989), laser desorption time-of-flight mass spectrometers (Karas and Hillenkamp, Anal. Chem. 60:2299-2301, 1988), and a Fourier Transform Ion Cyclotron Resonance Mass Spectrometer (Extrel Corp., Pittsburgh, Mass.).

MALDI ionization is a technique in which samples of interest are co-crystallized with an acidified matrix. The matrix is typically a small molecule that absorbs at a specific wavelength, generally in the ultraviolet (UV) range, and dissipates the absorbed energy thermally. Typically a pulsed laser beam is used to transfer energy rapidly (i.e., a few ns) to the matrix. This transfer of energy causes the matrix to rapidly dissociate from the MALDI plate surface and results in a plume of matrix and the co-crystallized analytes being transferred into the gas phase. MALDI is considered a “soft-ionization” method that typically results in singly-charged species in the gas phase, most often resulting from a protonation reaction with the matrix. MALDI may be coupled in-line with time of flight (TOF) mass spectrometers. TOF detectors are based on the principle that an analyte moves with a velocity proportional to its mass. Analytes of higher mass move slower than analytes of lower mass and thus reach the detector later than lighter analytes. The present disclosure contemplates methods of evaluating MALDI spectra obtained on a plurality of molecules and a matrix suitable for mass spectrometry. In certain instances, the matrix is a nicotinic acid derivative or a cinnamic acid derivative.

MALDI-TOF MS is easily performed with modern mass spectrometers. Typically the samples of interest are mixed with a matrix and spotted onto a polished stainless steel plate (MALDI plate). Commercially available MALDI plates can presently hold up to 1536 samples per plate. Once spotted with sample, the MALDI sample plate is then introduced into the vacuum chamber of a MALDI mass spectrometer. The pulsed laser is then activated and the mass to charge ratios of the analytes are measured utilizing a time of flight detector. A mass spectrum representing the mass to charge ratios of the peptides/proteins is generated.

MALDI can be utilized to measure the mass to charge ratios of both proteins and smaller peptide fragments. In the case of proteins, a mixture of intact protein and matrix can be co-crystallized on a MALDI target (Karas, M. and Hillenkamp, F. Anal. Chem. 1988, 60 (20) 2299-2301). The spectrum resulting from this analysis is employed to determine the molecular weight of a whole protein. This molecular weight can then be compared to the theoretical weight of the protein and utilized in characterizing the analyte of interest, such as whether or not the protein has undergone post-translational modifications (e.g., phosphorylation).

In certain embodiments of the method of the present disclosure, MALDI mass spectrometry can be used to evaluate peptide maps of proteins that have been fragmented, for example, by radiolysis, chemical or enzymatic reactions, etc. The peptide masses are measured accurately using a MALDI-TOF or a MALDI-Q-Star mass spectrometer, with detection precision down to the low ppm (parts per million) level. Evaluation of peptide maps by methods of the present disclosure can be useful in comparing mutants of the same protein, comparing a plurality of proteins or fragments from a plurality of proteins, comparing the binding of molecules to proteins, etc.

In certain embodiments, the present disclosure contemplates evaluating spectra collected by mass spectrometry for the purpose of screening molecules that bind a protein of interest. Such a method can comprise, (a) generating a purified protein of interest or fragment thereof; (b) preparing a sample of the protein and molecule in an appropriate solvent; (c) subjecting the protein bound to molecule to degradation, for example, by radiolysis, chemical or enzymatic reactions, etc. (d) subjecting the degraded sample to mass spectrometry, and (e) repeating (a) through (d) for a variety of molecules, thereby generating a plurality of spectra for evaluation by the methods of the present disclosure. In such an embodiment the attributes of the spectral parameter of number of peaks can include a fragmentation pattern and can be indicative of binding of the molecule to a particular surface of the protein. In such an embodiment the attributes of the spectral parameter of peak location can include mass and charge of a particular fragment and can be indicative of cleavage points along the polypeptide backbone. In certain embodiments, a training set of spectra collected on a plurality of molecules is evaluated using attributes of spectral parameters indicative of binding a specific surface of a protein (for example, a particular domain, side chain, etc.). In further embodiments, sample spectra are classified using attributes of spectral parameters indicative of binding to molecules thereby resulting in identification of samples that bind specific surfaces of a protein.

(iii) Evaluation of Spectra Collected by Infrared and RAMAN Spectrometry

In one embodiment, the present disclosure contemplates evaluating spectra acquired by the technique of infrared spectroscopy. In a further embodiment, the present disclosure contemplates evaluating spectra acquired by the technique of RAMAN spectroscopy.

In certain embodiments, the system and methods of the present disclosure can be used in order to characterize structural properties of molecules by the evaluation of spectra acquired by infrared or RAMAN spectroscopy. In such an embodiment, purified molecules can be made and subjected to spectroscopic analysis, thereby acquiring spectra appropriate for evaluation. Such an embodiment can comprise, (a) generating a purified molecule of interest, for example, a protein, nucleic acid, or small molecule, or a fragment thereof; (b) preparing a sample of the molecule in an appropriate solution; (c) subjecting the sample to spectroscopic analysis, and (d) repeating (a) through (c) for a variety of molecules, thereby acquiring a plurality of spectra for evaluation by the present disclosure. In certain embodiments, a training set of spectra collected on a plurality of molecules is evaluated using attributes of spectral parameters indicative of a particular structural property of the molecules (for example, orientation of a hydrogen bond, orientation of a side chain, etc.). In further embodiments, sample spectra are classified using attributes of spectral parameters indicative of a particular structural property of the molecules thereby resulting in identification of samples with said structural properties.

In certain embodiments, one can use the system and methods of the present disclosure in order to monitor the interaction between a selected molecule, for example, a protein, nucleic acid, or small molecule and one or more molecules by evaluating spectra acquired by the techniques of infrared or RAMAN spectroscopy. In such an embodiment, the disclosure can include detecting, designing and characterizing interactions between a selected molecule and test molecules, including proteins, nucleic acids and small molecules, utilizing infrared or RAMAN spectroscopy. For example, the present disclosure contemplates evaluating spectra obtained on a plurality of conditions while a molecule is in a complex with another molecule. Such an embodiment can comprise: (a) generating a purified molecule of interest, for example, a protein, nucleic acid or small molecule, or a fragment thereof; (b) forming a complex between the molecule and the test molecule; (c) subjecting the complex to spectroscopic analysis, and (d) repeating (a) through (c) for a variety of conditions, thereby acquiring a plurality of spectra for evaluation by the methods of the present disclosure. In certain embodiments, a training set of spectra collected on a plurality of conditions is evaluated using attributes of spectral parameters indicative of complex formation between the molecule of interest and another molecule. In further embodiments, sample spectra are classified using attributes of spectral parameters indicative of complex formation thereby resulting in identification of conditions which facilitate binding between the molecule of interest and another molecule.

In another example, the present disclosure contemplates evaluating spectra for identifying test molecules that bind to a molecule of interest (for example, a protein, nucleic acid or small molecule or a fragment thereof) utilizing infrared or RAMAN spectroscopy. Such an embodiment can comprise: (a) generating a purified molecule of interest, for example, a protein, nucleic acid or small molecule, or a fragment thereof; (b) forming a complex between the molecule and the test molecule; (c) subjecting the complex to spectroscopic analysis, and (d) repeating (a) through (c) for a variety of test molecules, thereby acquiring a plurality of spectra for evaluation by the methods of the present disclosure. In certain embodiments, a training set of spectra collected on a plurality of test molecules is evaluated using attributes of spectral parameters indicative of complex formation between the molecule of interest and another molecule. In further embodiments, sample spectra are classified using attributes of spectral parameters indicative of complex formation thereby resulting in identification of molecules that bind the molecule of interest.

Briefly, infrared and RAMAN spectroscopy measure part of the light propagated through a uniform material which is absorbed and transmitted by the molecule. The transmission of light by the molecule can be a scattering processes due to molecular vibrations. Infrared spectroscopy describes vibrational frequencies together with information concerning the absorption intensity. Infrared spectroscopy relies upon a change in the dipole moment of the absorbing species during a vibrational cycle. Since asymmetric species have larger dipole moments than more symmetric species, strong infrared spectral features arise from polarized groups and antisymmetric vibrations of symmetric groups. Raman scattering intensity depends upon the degree of modulation of the polarizability of the scattering species during a vibrational cycle. RAMAN frequencies arise from changes in the electronic polarizability associated with nuclear vibrational displacements. Thus symmetric vibrational modes of symmetric species and groups which contain polarizable atoms such as sulfur tend to scatter strongly. The three-dimensional structure and the intramolecular-intermolecular interactions of a molecule determine the frequencies and forms of its normal modes of vibration.

Infrared and RAMAN spectroscopy are useful techniques in generating unique fingerprint information for molecules. Information regarding the molecular structure of molecules can be determined by analyzing the attributes of the spectral parameters, for example, frequencies, intensities, and polarization states observed in Infrared and RAMAN spectra. Among the various molecules and characteristics that can be studied by infrared and RAMAN are, for example, protein conformational changes, charge effects, bond distortions, chemical rearrangement, phosphodiester backbone geometries of DNA and RNA; binding of test molecules to protein, nucleic acids or small molecules of interest, etc.

In embodiments of the present disclosure the spectral parameter of peak intensity in infrared or RAMAN spectra can be indicative of sample characteristics, for example, binding of a another molecule to the molecule of interest, chemical environment of a particular atom in a molecule of interest, etc. In further embodiments the spectral parameter of peak location can be indicative of sample characteristics, for example, structural features of the molecule of interest (for example, orientation of a bond, formation of a particular bond, etc.), binding of a molecule to the molecule of interest, etc.

### EXEMPLIFICATION

The disclosure now being generally described, it will be more readily understood by reference to the following examples which are included merely for purposes of illustration of certain aspects and embodiments of the present disclosure, and are not intended to limit the disclosure in any way.

### Example 1

### Protein Purification of ¹⁵N Labeled Polypeptides

The cells, harboring a plasmid each with a nucleic acid encoding a polypeptide of the invention, are inoculated into 2 L of M9 minimal media (containing ¹⁵N isotope, 0.48 g/L ¹⁵NH₄Cl) in a 6 L Erlenmeyer flask. The minimal media is supplemented with 0.01 mM ZnSO₄, 0.1 mM CaCl₂, 1 mM MgSO₄, 5 mg/L Thiamine.HCl, and 0.4% glucose. The 2 L culture is grown at 37° C. and 200 rpm to an OD₆₀₀ of between 0.7-0.8. The cultures are then induced with 0.5 mM IPTG in each culture and allowed to shake at 15° C. for 14 hours. The cells are harvested by centrifugation and the cell pellets are resuspended in 15 mL cold binding buffer each and 100 μl of protease inhibitor and flash frozen. The protein is then purified as described below from each of resuspended pellets.

Alternatively, the freshly transformed cells, harboring a plasmid each with the gene of interest, is inoculated into 10 mL of M9 media (with ¹⁵N isotope) and supplemented with 0.01 mM ZnSO₄, 0.1 mM CaCl₂, 1 mM MgSO₄, 5 mg/L Thiamine.HCl, and 0.4% glucose. After 8-10 hours of growth at 37° C., the cultures are transferred to a 2 L Baffled flask (Corning) containing 990 mL of the same media. When OD₆₀₀ of the culture is between 0.7-0.8, protein production is initiated by adding IPTG to a final concentration of 0.8 mM in each culture and lowering the temperature to 25° C. After 4 hours of incubation at this temperature, the cells are harvested, and the cell pellets are resuspended in 10 mL cold binding buffer (Hepes 50 mM, pH 7.5, 5% glycerol (v/v), 0.5 M NaCl, 5 mM imidazole) each and 100 μl of protease inhibitor and flash frozen.

The frozen pellets are thawed and sonicated to lyse the cells (5×30 seconds, output 4 to 5, 80% duty cycle, in a Branson Sonifier, VWR). The lysates are clarified by centrifugation at 14,000 rpm for 60 min at 4° C. to remove insoluble cellular debris. The supernatants are removed and supplemented with 1 μl of Benzonase Nuclease (25 U/μl, Novagen).

The recombinant protein is purified using DE52 (anion exchanger, Whatman) and Ni-NTA columns (Qiagen). The DE52 columns (30 mm wide, Biorad) are prepared by mixing 10 grams of DE52 resin in 25 ml of 2.5 M NaCl per protein sample, applying the resin to the column and equilibrating with 30 ml of binding buffer (50 mM in HEPES, pH 7.5, 5% glycerol (v/v), 0.5 M NaCl, 5 mM imidazole). Ni-NTA columns are prepared by adding 3.5-8 ml of resin to the column (20 mm wide, Biorad) based on the level of expression of the recombinant protein and equilibrating the column with 30 ml of binding buffer. The columns are arranged in tandem so that the protein sample is first passed over the DE52 column and then loaded directly onto the Ni-NTA column.

The Ni-NTA columns are washed with at least 150 ml of wash buffer (50 mM HEPES, pH 7.5, 5% glycerol (v/v), 0.5 M NaCl, 30 mM imidazole) per column. A pump may be used to load and/or wash the columns. The protein is eluted off of the Ni-NTA column using elution buffer (50 mM in HEPES, pH 7.5, 5% glycerol (v/v), 0.5 M NaCl, 250 mM imidazole) until no more protein is observed in the aliquots of eluate as measured using Bradford reagent (Biorad). The eluate is supplemented with 1 mM of EDTA and 0.2 mM DTT.

The samples are assayed by SDS-PAGE and stained with Coomassie Blue, with protein purity determined by visual staining.

### Example 2

### Acquisition of HSQC NMR Spectra on Multiple Proteins

NMR experiments were performed on a Bruker Avance 600-MHz spectrometer equipped with a 5-mm triple-resonance cryo-probe head at 298 K. NMR samples were typically prepared in 500 μl of 90% H₂O-10% D₂O buffer containing 500 mM NaCl, 10 mM HEPES buffer at pH 7.5 (pH reading is not corrected for isotopic effects). The two dimensional (¹H, ¹⁵N)HSQC experiments were acquired using the pulse sequence described by Davis et al. (J. Magn. Reson. 98: 207-216 (1992)), Grzesiek and Bax (J. Am. Chem. Soc. 115: 12593-12594 (1993)) with water suppression by flip-back pulses. The sweep width was 14 ppm and 45 ppm in the ¹H and ¹⁵N dimensions, respectively. The ¹H carrier was set at 600.1324 MHz, while the ¹⁵N carrier at 60.1778 MHz. The size of the HSQC spectra gave a 1024×128 real data matrix. The spectra were processed on a Ultra 5 computer from SUN Microsystems using NMRPipe software (Delaglio et al., J. Biomol. NMR 6: 277-293 (1995)).

### Example 3

### Manual Categorization of HSQC NMR Spectra of the Training Set

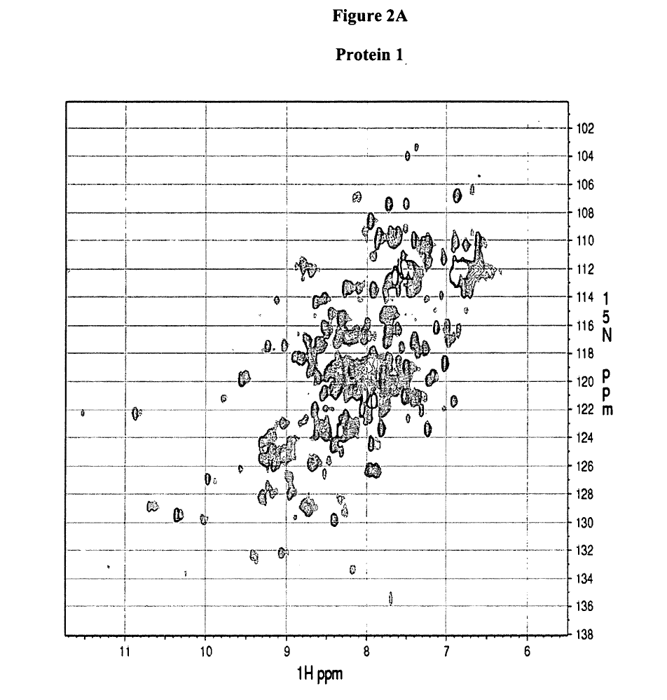

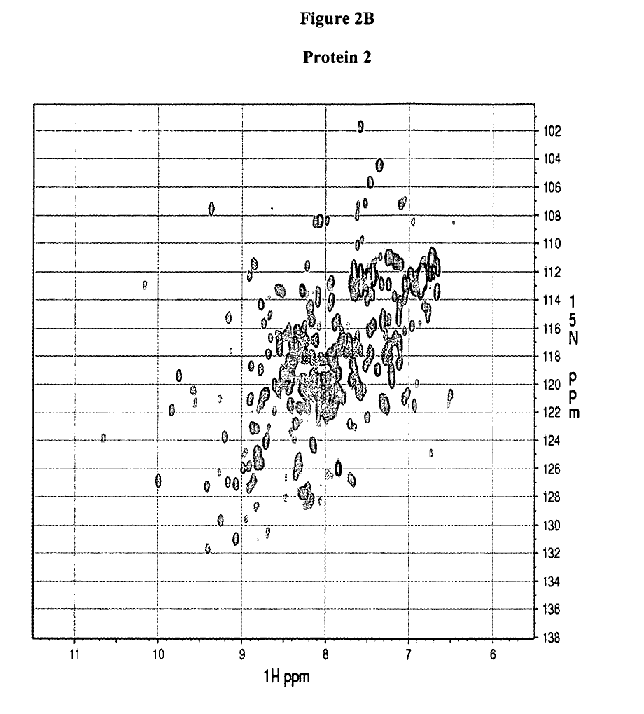

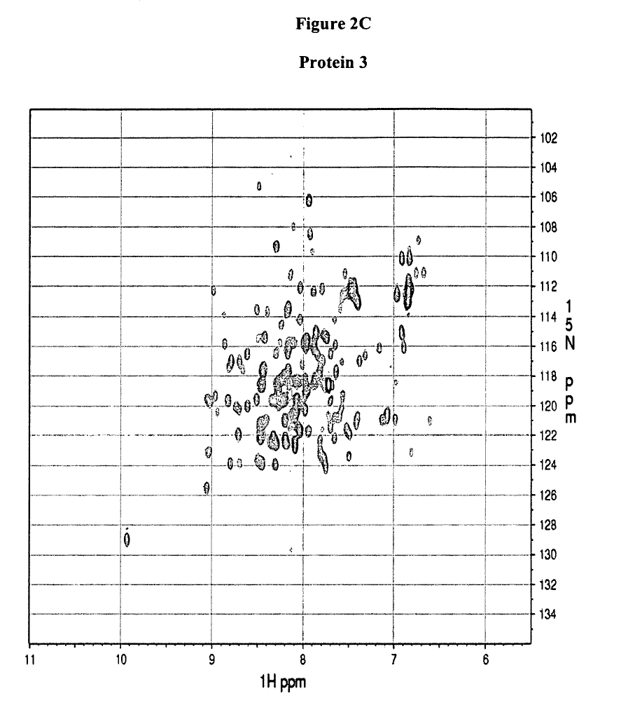

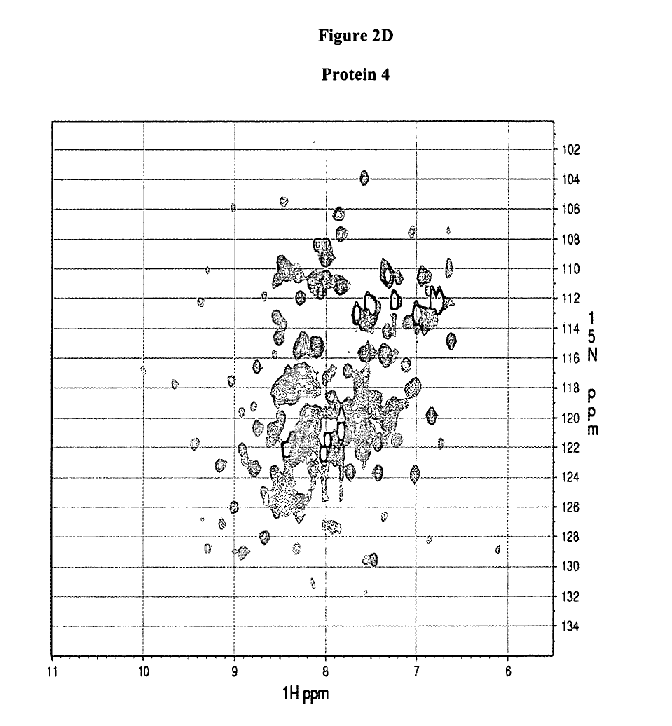

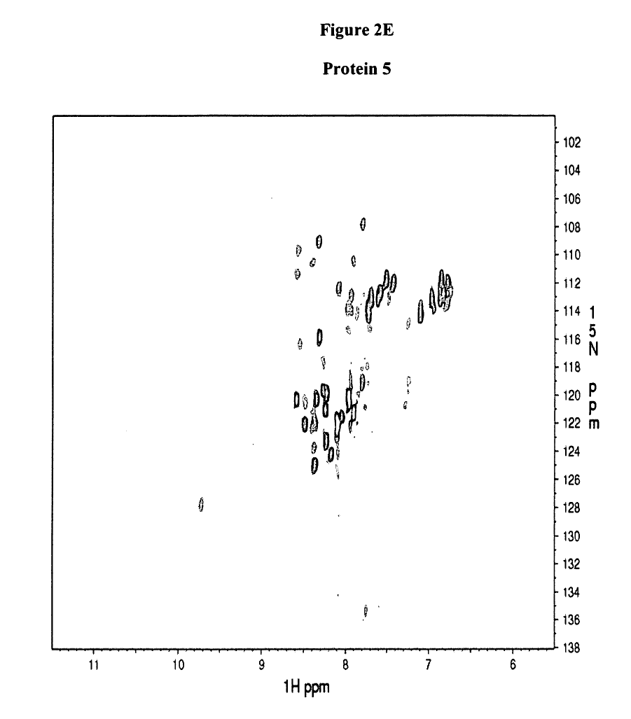

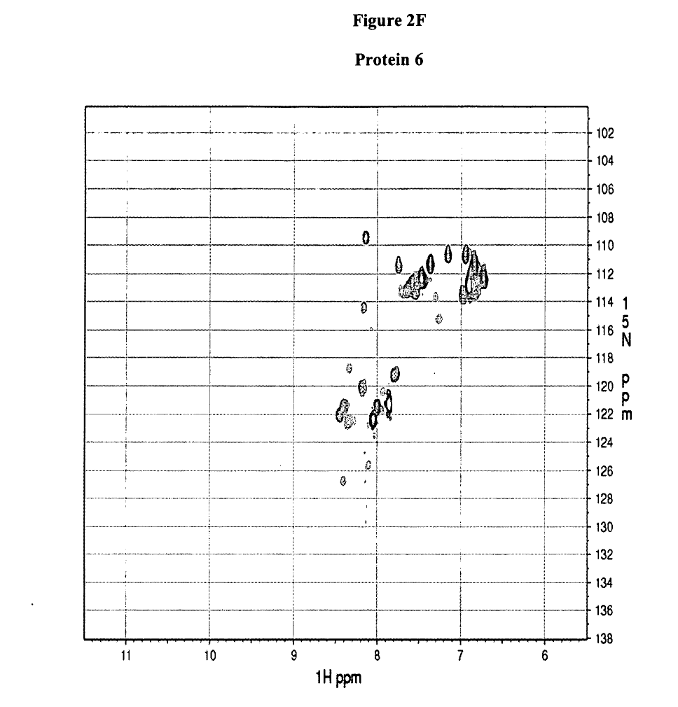

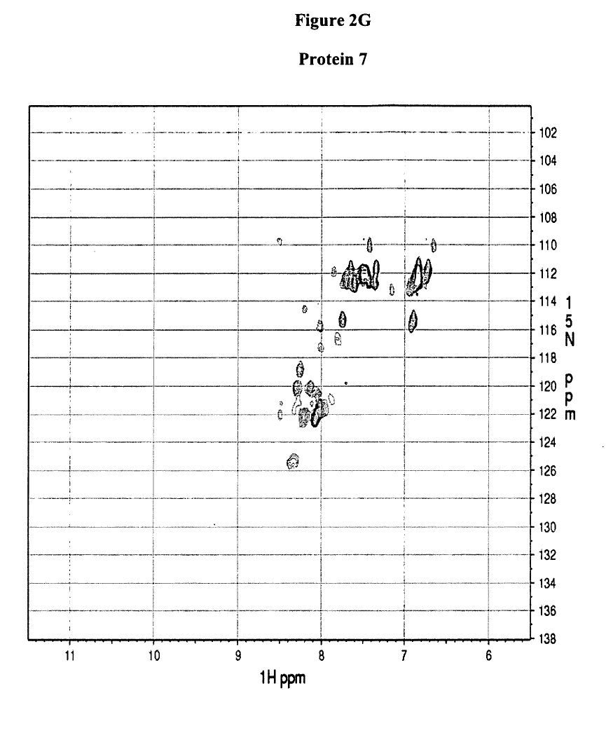

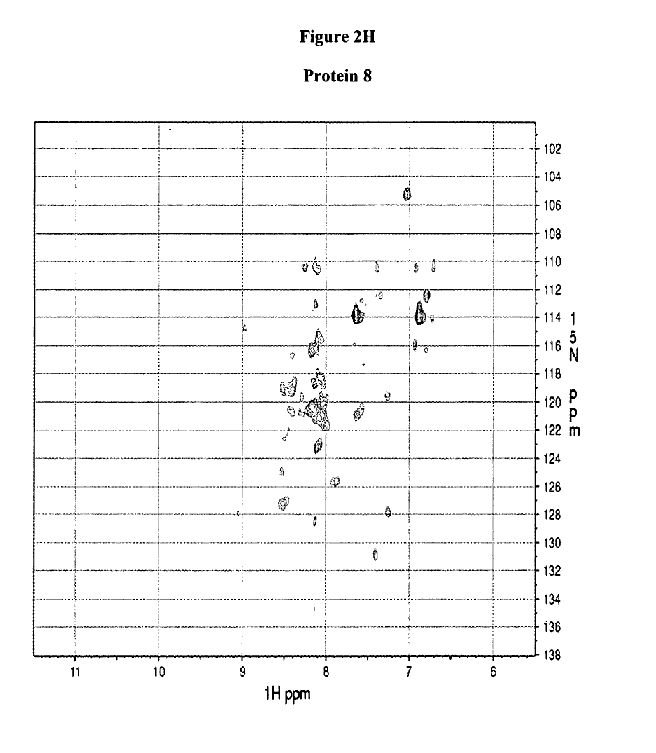

The protein spectra obtained in Example 2 (FIG. 2) are associated with one of the following four categories: (a) good, Protein 1 and 2, (b) promising, Protein 3 and 4, (c) unfolded, protein 5 and 6, and (d) poor, Protein 7 and 8. The association takes into account the chemical shift dispersion in both proton and nitrogen dimension, intensity and line-width of the peaks and number of peaks observed versus the number of peaks expected which equals the number of non-proline residues in the protein (excluding side chain NH₂ groups). Typically, a ¹H chemical shift range from 5.5 to 12 ppm and a ¹⁵N chemical shift range from 98 to 140 ppm is considered.

TABLE 1

| HSQC spectra of | Classification |
|---|---|
| Protein 1 | Good |
| Protein 2 | Good |
| Protein 3 | Promising |
| Protein 4 | Promising |
| Protein 5 | Unfolded |
| Protein 6 | Unfolded |
| Protein 7 | Poor |
| Protein 8 | Poor |

### Example 4

### Evaluation of the Training Set of HSQC NMR Spectra

The following attributes were considered to evaluate the spectra (FIG. 2) associated with categories in Table 1: (i) peak positions in the proton and nitrogen dimensions, (ii) the fraction of the expected peaks that were observed, and (iii) peak width and peak intensity. Peak positions were estimated in proton and nitrogen chemical shift by multidimensional parabolic models within the NMRPipe software (Delaglio et al., J. Biomol. NMR 6: 277-293 (1995)). A two-dimensional vector of means was established using an unbiased variance-covariance matrix of peak positions. Picking and counting peaks yielded the observed fraction of expected peaks parameter. The fraction of observed peaks was calculated as the number of observed peaks divided by the number of the theoretically expected peaks (the number of non-proline residues in the protein). Peak intensity was defined by parameters of the intensity distribution which are invariant with respect to the distribution's rescaling. Line width and extent of the peak in all dimensions was measured by multidimensional parabolic models within the NMRPipe software

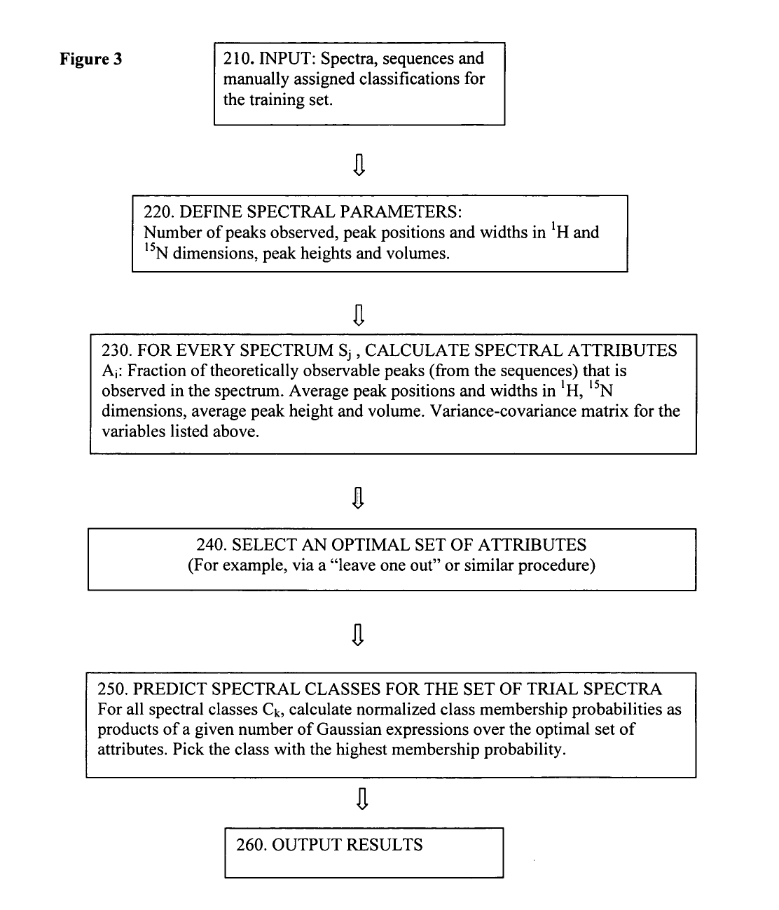

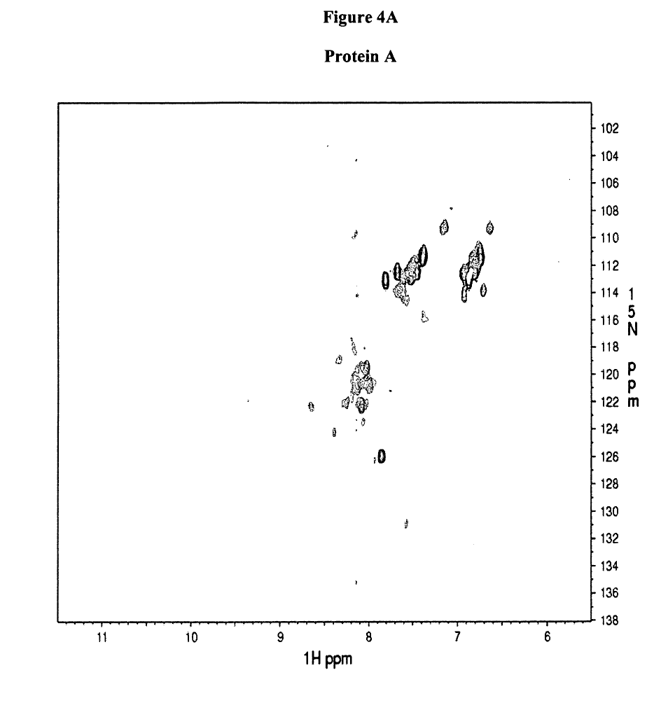

The algorithm in FIG. 3 consists of 2 main stages. During the first stage, an optimal set of the attributes is automatically selected by minimizing a “leave one out” classification rate via a greedy search procedure over all unique combinations of up to 3 attributes. In the second stage an optimized classifier, obtained as specified above, is used to predict the classes of the cases within the test data set. An example of the leave one out procedures is as follows:

For all test spectra Sⱼ:

For all single attributes (Aᵢ):
For all spectral classes Cₖ:
   Calculate average and standard deviation for the attribute value, over all spectra except Sᵢ: AVER(Aᵢ|Cₖ, excl Sⱼ), STDEV(Aᵢ|Cₖ, excl Sⱼ).
Predict spectral class for Sⱼ:
   For all spectral classes Cₖ:, calculate normalized class membership probabilities as Gaussian expressions on AVER(Aᵢ|Cₖ, excl Sⱼ), STDEV(Aᵢ|Cₖ, excl Sⱼ)
   Pick the class with the highest membership probability.
   Check correctness of the prediction with respect to the manually assigned class. If incorrect, add 1 to the incorrect predictions count, N_{INCORR}(Aᵢ)

For all unique pairs of attributes (Aᵢ₁, Aᵢ₂):
For all spectral classes Cₖ:
   Calculate average and standard deviation for the attribute value, over all spectra except Sᵢ: AVER(Aᵢ₁|Cₖ, excl Sⱼ), STDEV(Aᵢ₁|Cₖ, excl Sⱼ), AVER(Aᵢ₂|Cₖ, excl Sⱼ), STDEV(Aᵢ₂, Cₖ, excl Sⱼ)
Predict spectral class for Sⱼ:
   For all spectral classes Cₖ:, calculate normalized class membership probabilities as products of two Gaussian expressions on AVER(Aᵢ₁|Cₖ, excl Sⱼ), STDEV(Aᵢ|Cₖ, excl Sⱼ) and AVER(Aᵢ₂|Cₖ, excl Sⱼ), STDEV(Aᵢ₂|Cₖ, excl Sⱼ)
   Pick the class with the highest membership probability.
Check correctness of the prediction with respect to the manually assigned class. If incorrect, add 1 to the incorrect predictions count, N_{INCORR}(Aᵢ₁, Aᵢ₂)

For all unique triplets of attributes (Aᵢ₁, Aᵢ₂, Aᵢ₃):
For all spectral classes Cₖ:
   Calculate average and standard deviation for the attribute value, over all spectra except Sᵢ: AVER(Aᵢ₁|Cₖ, excl Sⱼ), STDEV(Aᵢ₁|Cₖ, excl Sⱼ), AVER(Aᵢ₃|Cₖ, excl Sⱼ), STDEV(Aᵢ₃, Cₖ, excl Sⱼ).
Predict spectral class for Sⱼ:
   For all spectral classes Cₖ, calculate normalized class membership probabilities as products of three Gaussian expressions on AVER(Aᵢ₁|Cₖ, excl Sⱼ), STDEV(Aᵢ₁|Cₖ, excl Sⱼ), and AVER(Aᵢ₂, Cₖ, excl Sⱼ), STDEV(Aᵢ₂|Cₖ, excl Sⱼ), and AVER(Aᵢ₃, Cₖ, excl Sⱼ), STDEV(Aᵢ₃|Cₖ, excl Sⱼ)
   Pick the class with the highest membership probability.
Check correctness of the prediction with respect to the manually assigned class. If incorrect, add 1 to the incorrect predictions count, N_{INCORR}(Aᵢ₁, Aᵢ₂, Aᵢ₃)
For all single attributes (Aᵢ), unique pairs of attributes (Aᵢ₁, Aᵢ₂), and unique triplets of attributes (Aᵢ₁, Aᵢ₂, Aᵢ₃):
   Select the combination with a minimal N_{INCORR} as an optimal set of attributes for further predictions.

### Example 5

### Evaluation of the Sample HSQC NMR Spectra

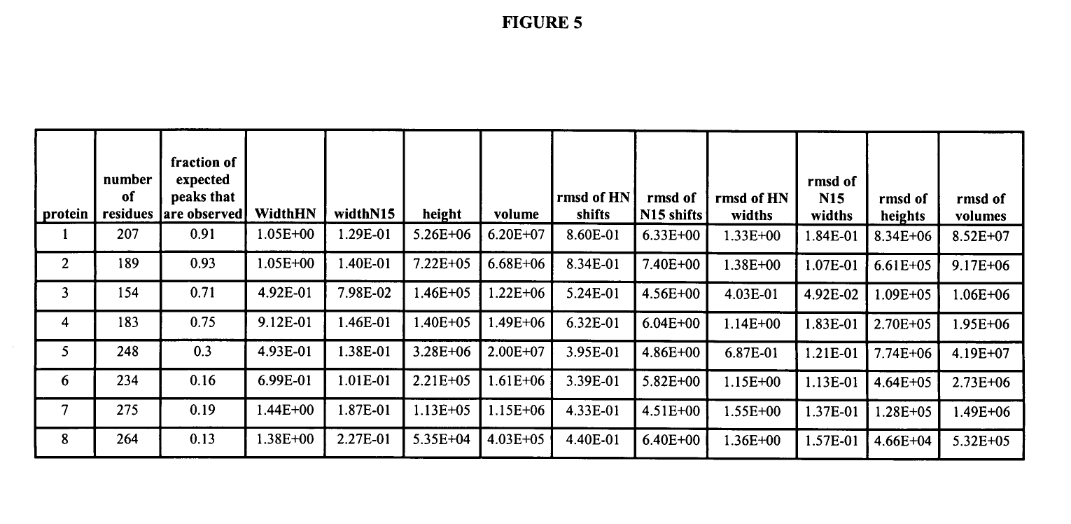

The spectra in EXAMPLE 2 (FIG. 2) were automatically evaluated. The results are shown in Table 2 presented in FIG. 5.

The evaluation results as membership probabilities using method described in Example 4 are shown below.

TABLE 3

predicted class membership probabilities

| protein | good | prom | unfo | poor |
|---|---|---|---|---|
| 1 | 1 | 0 | 0 | 0 |
| 2 | 0.999 | 0.001 | 0 | 0 |
| 3 | 0.017 | 0.983 | 0 | 0 |
| 4 | 0.41 | 0.59 | 0 | 0 |
| 5 | 0 | 0.004 | 0.996 | 0 |
| 6 | 0 | 0.002 | 0 | 0.998 |
| 7 | 0 | 0.015 | 0 | 0.985 |
| 8 | 0 | 0.002 | 0 | 0.998 |

The results form manual and automatic evaluation are compared in the table below. These two sets of results agree well with each other. In practice, manual evaluation is performed arbitrarily according to the experience of those with skills in the art. Therefore, manual evaluation tends to lose its consistency and accuracy if it is performed by a different person, or if a number of samples are evaluated different times by the same person. Automatic evaluation offers a systematic method with greater accuracy, which is always consistent.

TABLE 4

| protein | manual evaluation | automatic evaluation |
|---|---|---|
| 1 | good | good |
| 2 | good | good |
| 3 | prom | promising |
| 4 | promising | promising |
| 5 | unfolded | unfolded |
| 6 | unfolded | poor |
| 7 | poor | poor |
| 8 | poor | poor |

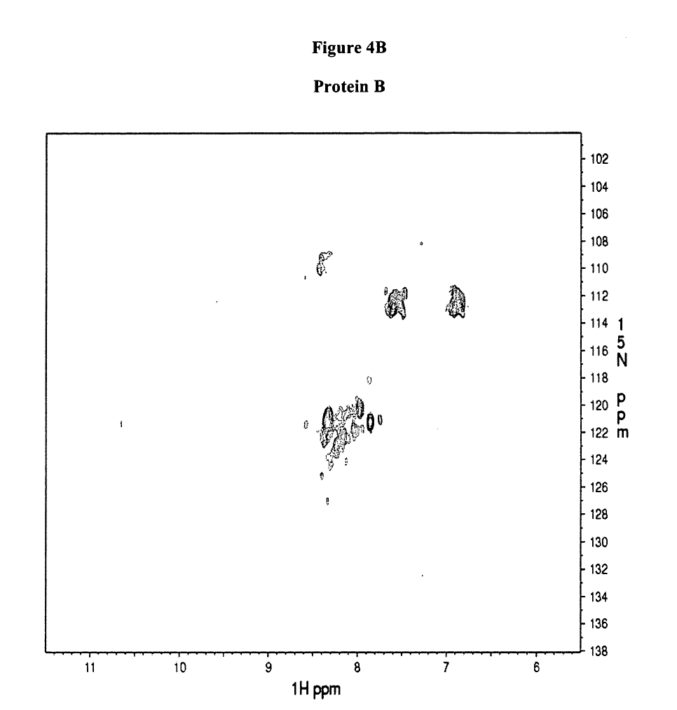

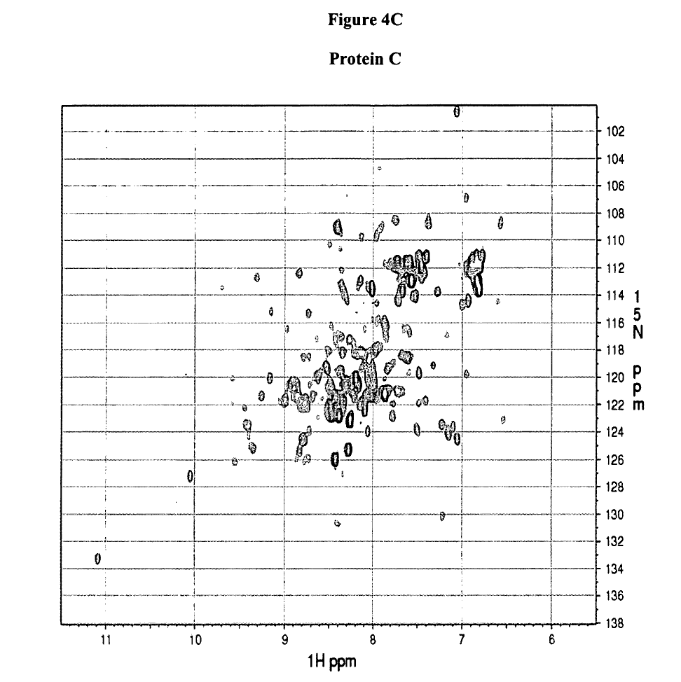

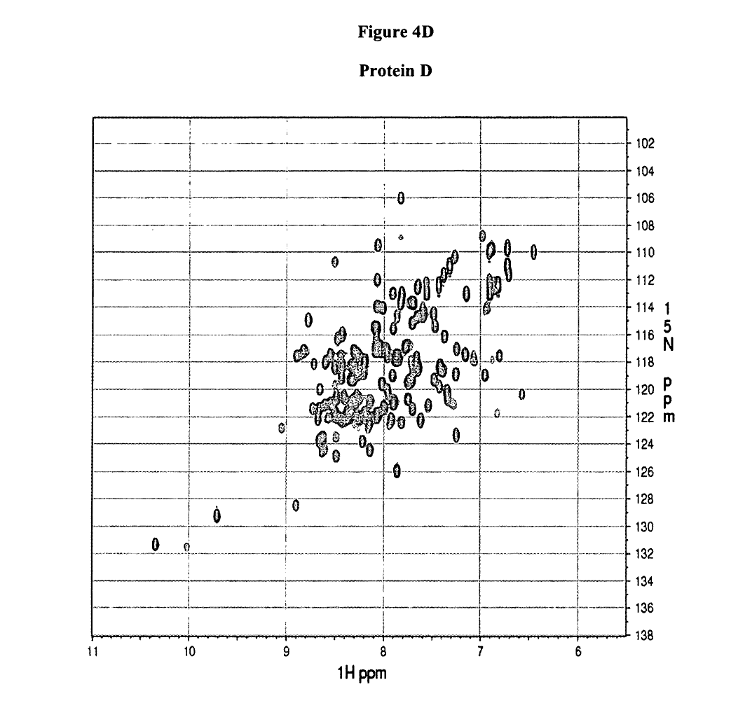

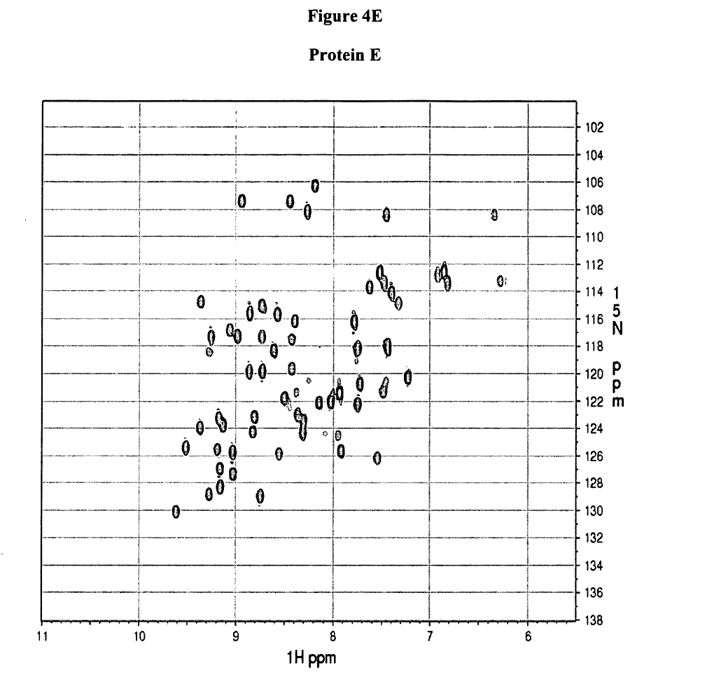

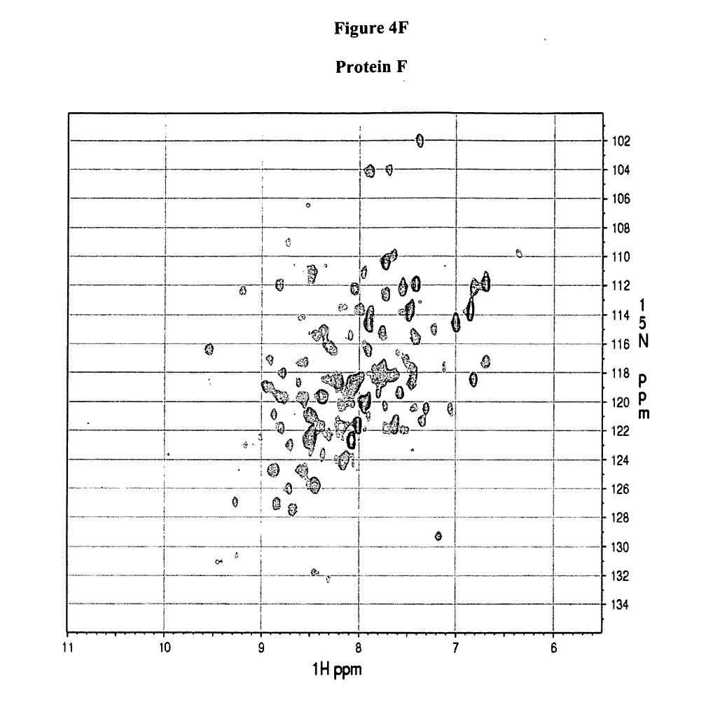

Additional spectra presented in FIG. 4 were also evaluated as explained here for proteins 1-8. For the spectra in FIG. 4 labeled A-F, the results of automatic evaluation are shown below in Table 5. The comparison of results from manual and automatic evaluation are shown below in Table 6.

TABLE 5

predicted class membership probabilities

| Protein | good | prom | unfo | poor |
|---|---|---|---|---|
| A | 0 | 0.004 | 0 | 0.996 |
| B | 0 | 0 | 0 | 1 |
| C | 0.005 | 0.995 | 0 | 0 |
| D | 0.858 | 0.142 | 0 | 0 |
| E | 0.31 | 0.69 | 0 | 0 |
| F | 0.456 | 0.544 | 0 | 0 |

TABLE 6

| protein | manual evaluation | automatic evaluation |
|---|---|---|
| A | unfolded | poor |
| B | poor | poor |
| C | promising | promising |
| D | good | good |
| E | good | promising |
| F | promising | promising |

Equivalents

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference are the following: Albala et al. (2000) Journal of Cellular Biochemistry 80: 187-191; Feng et al. (2001) Analytical Chemistry 73: 5691-5697; Draveling et al. (2001) Protein Expression and Purification 22: 359-366); Eberstadt et al. (1998) Nature 392: 941-945; Krafft et al. (1998) Biophysical Journal 74, 63-71; Surewica et al. (1988) Biochimica et Biophysica Acta 952: 115-130; U.S. Pat. Nos. 5,940,307; 6,040,191; 5,668,734; 6,194,179; 6,162,627; 6,043,024; 5,817,474; 5,891,642; 5,989,827; 5,891,643; 6,077,682; WO 00/05414; WO 99/22019.

What is claimed is:

1. A method of evaluating one or more spectra, comprising:
(i) providing a training set based on a plurality of spectra;
(ii) associating said spectra of said training set based on the attributes of at least two spectral parameters with at least two categories;
(iii) scoring said at least two spectral parameters of said spectra of said training set in the at least two categories;
(iv) comparing the spectral parameters of one or more sample spectra to said scored spectral parameters of said training set; and
(v) classifying said one or more sample spectra into one of said categories based on the comparison.

2. The method of claim 1, wherein said spectra are NMR spectra.

3. The method of claim 2, wherein said spectral parameters include at least one of the following: chemical shift, ratio of observed peaks to expected peaks, and peak intensity.

4. The method of claim 2, wherein said NMR spectra of said training set and said one or more sample NMR spectra are obtained on samples comprising protein.

5. The method of claim 2, wherein said NMR spectra of said training set and said one or more sample NMR spectra are obtained on samples comprising nucleic acid.

6. The method of claim 2, wherein said NMR spectra of said training set comprise a two-dimensional spectrum.

7. The method of claim 2, wherein said comparing the spectral parameters of one or more sample NMR spectra to said scored spectral parameters of said training set comprises using a statistical approach comprising a Bayesian classifier for at least one of said spectral parameters.

8. The method of claim 2, wherein said comparing the spectral parameters of one or more sample NMR spectra to said scored spectral parameters of said training set comprises computing a probability distribution for at least one of said spectral parameters.

9. The method of claim 2, wherein said comparing the spectral parameters of one or more sample NMR spectra to said scored spectral parameters of said training set comprises using a statistical approach comprising neural networks for at least one of said spectral parameters.

10. A method of evaluating a plurality of spectrum, comprising: (i) providing a training set based on a plurality of spectrum; (ii) associating said spectra of said training set based on the attributes of at least two spectral parameters with at least two categories; (iii) scoring said spectral parameters of said spectra of said training set into said categories.

11. The method of claim 10, wherein said spectra are NMR spectra.

12. The method of claim 10, wherein said spectral parameters include at least one of the following: chemical shift, ratio of observed peaks to expected peaks, and peak intensity.

13. The method of claim 10, wherein said NMR spectra of said training set and said sample NMR spectrum are obtained on samples comprising protein.

14. The method of claim 10, wherein said NMR spectra of said training set and said sample NMR spectrum are obtained on samples comprising nucleic acid.

15. The method of claim 10, wherein said NMR spectra are two-dimensional spectra.

16. A method of evaluating one or more sample spectra, comprising:
(i) obtaining a training set of a plurality of spectrum scored by the attributes of at least two or more spectral parameters in two or more categories,
(ii) comparing the spectral parameters of one or more sample spectra to said scored spectral parameters of said training set; and
(iii) classifying said one or more sample spectra into said categories based on the results of such comparison.

17. The method of claim 16, wherein said spectra are NMR spectra.

18. The method of claim 17, wherein said spectral parameters include at least one of the following: chemical shift, ratio of observed peaks to expected peaks, and peak intensity.

19. The method of claim 17, wherein said NMR spectra of said training set and said sample NMR spectrum are obtained on samples comprising protein.

20. The method of claim 17, wherein said NMR spectra of said training set and said sample NMR spectrum are obtained on samples comprising nucleic acid.

21. The method of claim 17, wherein said NMR spectra are two-dimensional spectra.

22. The method of claim 17, wherein the comparing comprises using a statistical approach comprising a Bayesian classifier.

23. The method of claim 17, wherein the comparing comprises using a statistical approach comprising neural networks.

24. The method of claim 17, wherein the comparing comprises computing a probability distribution for said attribute of said spectral parameter.

25. A computer product for evaluating one or more sample NMR spectra, the computer product disposed on a computer-readable medium and having instructions for causing a processor to:
(i) score attributes of at least one spectral parameter of one or more NMR spectra associated with one or more categories of a training set;
(ii) compare said one or more spectral parameters of said one or more sample NMR spectra to the scored spectral parameters of said training set; and
(iii) classify said one or more sample NMR spectra into one of said categories.

26. The computer product of claim 25, wherein said spectral parameters include at least one of the following: chemical shift, ratio of observed peaks to expected peaks, and peak intensity.

27. The computer product of claim 25, wherein said NMR spectra are obtained on samples comprising protein.

28. The computer product of claim 25, wherein said NMR spectra are obtained on nucleic acid.

29. The computer product of claim 25, wherein said NMR spectra are two-dimensional spectra.

30. The computer product of claim 25, wherein said instructions to compare include instructions to use a statistical approach including a Bayesian classifier.

31. The computer product of claim 25, wherein said instructions to compare include instructions to use a statistical approach including neural networks.

32. The computer product of claim 25, wherein said instructions to compare comprise instructions to compute a probability distribution for said attribute of said spectral parameter.
